(19) United States
(12) Patent Application Publication
(10) Pub. No.: US 2003/0175326 A1
(43) Pub. Date: Sep. 18, 2003

(54) Palatable controlled-release formulations for companion animals

(76) Inventor: Avinash G. Thombre, East Lyme, CT (US)

Correspondence Address: PFIZER INC., PATENT DEPARTMENT, MS8260-1611, EASTERN POINT ROAD, GROTON, CT, 06340, US

(21) Appl. No.: 10/213,162

(22) Filed: Aug. 6, 2002

Related U.S. Application Data
(63) Continuation-in-part of application No. 10/091,202, filed on Mar. 5, 2002.

(51) Int. Cl. A23K 1/165; A23K 1/17; A61K 9/20

(52) U.S. Cl. 424/442

(57) ABSTRACT

The invention pertains to chewable controlled-release multiparticulate pharmaceutical compositions for companion animals and method of making same.

This is a continuation-in-part of U.S. Ser. No. 10/091,202, filed Mar. 5, 2002, pending, the entire contents of which are hereby incorporated by reference.

### FIELD OF THE INVENTION

The invention pertains to palatable controlled release pharmaceutical compositions for oral administration to companion animals. Advantageously, the compositions are chewable, there being no significant adverse affect on the controlled release behavior due to mastication. In particular, the compositions are comprised of pharmaceutically active agents in the form of controlled release multiparticulates, and a palatability improving agent.

### BACKGROUND OF THE INVENTION

Oral dosage forms of pharmaceuticals for companion animals, e.g. dogs and cats, have proven particularly valuable where the medication is to be administered on a chronic basis, especially by the pet owner. Typically, these dosage forms are of a size and shape that can accommodate administration by the “poke down” method whereby the medication is in the form of a tablet or the like placed on the animal's tongue or elsewhere in the mouth whereafter specific manipulations are performed by the handler to coax swallowing. Occasionally, if the taste of the medication is not intrinsically disfavorable, the animal will consume it of their own volition when proffered (the “free choice” method) without resort to the poke down technique. In these instances, however, it is common for the animal to chew the dosage form before swallowing it. While this generally bears no consequence to immediate release dosage forms, i.e., forms where no extrinsic factors delay the release of the pharmaceutically active agent, chewing is decidedly unsuitable for controlled release dosage forms known heretofore.

Controlled release dosage forms are those wherefrom the rate of release of the pharmaceutically active agent is controlled by an extrinsic factor related to the dosage form itself, such as specific coatings that erode, or through which the active agent must pass, thereby engendering a prolonged release pattern.

Therapeutically, controlled release dosage forms are desirable inasmuch as they can surmount pharmacokinetic limitations inherent in a particular active agent, such as unduly short half life. In other instances, they can also overcome food effects, i.e., situations where the rate and extent of active agent absorption depends on whether it was taken after eating (fed state) or after fasting (fasted state). Controlled release dosage forms have the further benefit that, in certain circumstances, they are able to reduce fluctuations in the plasma concentration of the active agent. For example, when plasma concentration fluctuates too low, the active agent can become ineffective, nullifying the therapy; when too high, unwanted side effects can manifest. Moreover, as compared to an immediate release dosage form, the peak plasma concentrations following a controlled release dosage form are lower, and the time to reach the peak plasma peak concentrations is longer, as is the apparent terminal half-life. Advantageously, this can result in less frequent dosing as well as a reduction in side effects associated with high drug concentrations in the gastrointestinal tract (local or topical side effects) and those associated with very high plasma concentrations (central effects). Additionally, controlled release dosage forms can also lower the dosage needed and reduce the total daily requirements of the active agent. Because of these attributes, therapy using controlled release dosage forms is often preferred over immediate release dosage forms.

Orally administered controlled release dosage forms are typically configured as controlled release matrix tablets. Conventionally, these tablets are fabricated by admixing the pharmaceutically active agent with a rate controlling polymer and optionally other ingredients (carriers and the like) whereafter they are pressed into tablet shape. The rate controlling polymer is ordinarily a hydrophilic or lipophilic polymer. Functionally, in the case of hydrophilic matrix tablets, when exposed to water or an aqueous environment, as when ingested, the polymer swells and forms a gel through which the active agent slowly diffuses out of the tablet. In addition to diffusion, the active agent is also released through the secondary mechanism of polymer erosion. In the case of lipophilic matrix tablets, the primary mechanism of release is via diffusion through pores in the matrix, which pores are formed by the leaching of the active agent or other water soluble inert ingredients incorporated into the tablets.

Chewing of a controlled release matrix tablet by a companion animal vitiates the very utility otherwise provided by this mode of dosage form. That is, the surface area of the dosage form is an important factor governing the rate of controlled release. Chewing breaks the dosage form, whose design has been predeterminedly predicated on surface area among other things, into pieces. This not only makes the particles of active agent smaller—hence increasing overall surface area and speeding up release rates—but also exposes more of the active agent from behind the polymer coatings—thus bypassing the control imposed by same—and otherwise reduces the distance between the active agent and the surface of the coating thereby diminishing diffusion times and the like. In an extreme case, the companion animal will chew the tablet to a powder, which effectively causes a complete loss of the rate controlling mechanism. In short, the controlled release dosage forms known hitherto for companion animals, when chewed, steadily loose their aforementioned performance attributes, and become increasingly like an immediate release dosage form, with all drawbacks of same.

Chewing of conventional controlled release dosage forms, such as matrix tablets, is especially aggravated where the tablet includes a palatability improving agent, the enhanced flavor from which can lead to even more enthusiastic mastication by the animal.

In addition to the problems attendant chewing, conventional controlled release dosages forms are typically provided as single unit dosage forms that can not be readily divided into a lesser dose wherein controlled rate of release is maintained. For example, oral therapy for companion animals frequently entails dosing on a milligram of dose per kilogram body weight basis; among other things, this accommodates the species-dependent variation in animal weight. Thus, it is important to be able to easily divide a tablet to obtain the most appropriate dose for a given animal of given weight. The alternative is to employ multiple dosages or tablet strengths, neither of which is practicable. However, if a controlled release matrix tablet is divided, the problems of breaking the dosage form into pieces, as elucidated above, attend. That is, the active agent can suffer reduced particle size, with resulting increase in surface area and release rate, as well as exposure from under, or a decrease in distance from the polymer coating whose presence is designed to control release rate.

While animals such as dogs and cats have been and are utilized as models in the development of controlled release drugs for humans in order to evaluate safety and performance characteristics of same, these efforts typically involve modes of administration wherein the chewing of the dosage form, e.g., the poke-down method, and the consequent adverse affect of destroying the controlled delivery mechanism, is not a concern. In addition, these practices do not entail the inclusion of palatability improving agents which could invite assertive chewing with attendant loss of controlled release.

Accordingly, there is a need for a controlled release dosage form that can be orally administered to a companion animal, which form can include a palatability agent and be chewed by the animal or divided without significant loss of the controlled release effect.

### SUMMARY OF THE INVENTION

The present invention satisfies the foregoing desiderata.

In one aspect, the invention is directed to a palatable, chewable, controlled release pharmaceutical composition for oral administration to a companion animal comprising a therapeutically effective amount of a pharmaceutically active agent in controlled release multiparticulate form; and a palatability improving agent in an amount sufficient to make the pharmaceutical composition palatable to said companion animal.

In another aspect, the invention is directed to a process for preparing a palatable, chewable, controlled release pharmaceutical composition for oral administration to a companion animal comprising preparing a therapeutically effective amount of a pharmaceutically active agent in the form of particles having an average particle size of up to about 5000 μm; coating said particles with a delayed release polymer, a sustained released polymer, or combinations of same, in an amount of about 5% to about 100% by weight of the pharmaceutical composition; admixing a palatability improving agent to said coated particles in an amount of about 0.025% to about 99% by weight of said pharmaceutical composition; and forming said admixture into a shape suitable for oral administration to a companion animal.

### BRIEF DESCRIPTION OF THE DRAWINGS

FIG. 1 is a graph showing the dissolution versus time of carprofen multiparticulates coated with various levels of Eudragit S100.

FIG. 2 is a graph showing the dissolution versus time using a pH cross over of uncoated and Eudragit S100-coated carprofen multiparticulates.

FIG. 3 is a graph showing dissolution versus time for carprofen multiparticulates in tablet form, uncoated and with various Eudragit S100 coatings.

FIG. 4 is a graph showing dissolution versus time using a pH cross over for carprofen multiparticulates in tablet form, uncoated and with various Eudragit S100 coatings.

FIG. 5 is a graph showing dissolution versus time of carprofen multiparticulates (microcapsule embodiment) with various coatings.

FIG. 6 is a graph showing dissolution versus time and the effect of tabletting and tablet hardness on carprofen microcapsules at a 25% coating level.

FIG. 7 is a graph showing plasma concentrations versus time in beagle dogs for 50mg immediate release carprofen multiparticulate formulations.

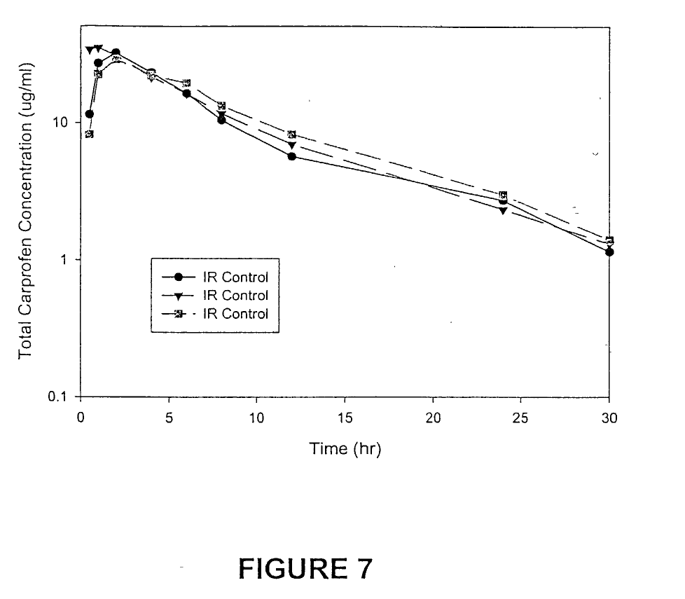

FIG. 8 is a graph showing plasma concentrations versus time in beagle dogs for delayed release carprofen multiparticulate formulations.

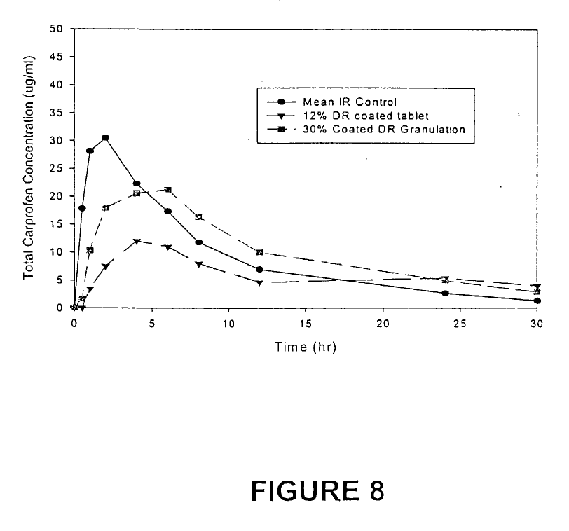

FIG. 9 is a graph showing plasma concentrations versus time in beagle dogs for sustained release carprofen multiparticulate formulations.

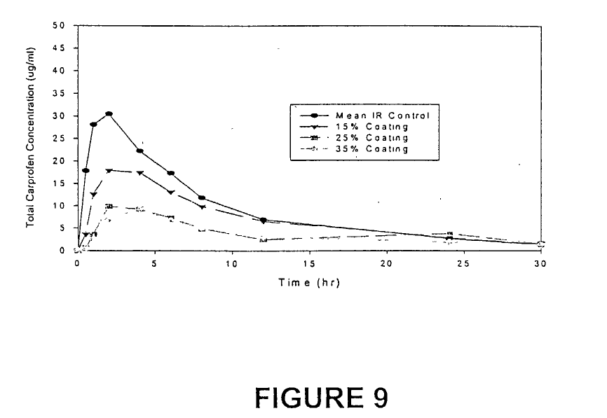

FIG. 10 is a graph showing plasma concentrations versus time in beagle dogs for compressed and uncompressed sustained release carprofen multiparticulate formulations.

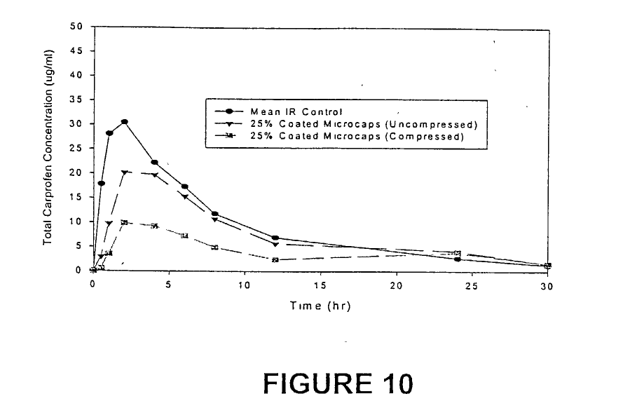

### DETAILED DESCRIPTION OF THE INVENTION

The present invention is directed to a controlled release pharmaceutical composition for companion animals that can be orally administered by veterinarian, pet owner or other caregiver. The composition of the invention is chewable, without the accrual thereby of any significant loss of the controlled release property. That is to say, the benefits associated with controlled release therapy as elucidated above are substantially maintained even after mastication by the animal. Thus it will be understood that chewable in the present context means that the controlled release performance of the dosage form is effectively resistant to chewing. Although the present invention specifically envisions the chewing of the composition thereof, it will be understood that the composition of the invention can also be administered by the “poke down” method aforesaid and that such administration is contemplated as being within the inventive scope. Thus for example if an animal is unable, by sickness or other factors, to accept medication by free choice, the poke down technique can be employed using the composition of the invention in a dosage form of suitable size and shape.

“Controlled release” (CR) as intended by the invention refers to the rate of release of a pharmaceutically active agent as a function of some property of the dosage form. Controlled release systems contemplated by the invention include without limitation modified systems such as 1) sustained release, wherein the pharmaceutically active agent is released at a slow rate over an extended period of time; 2) delayed release, wherein there is a time lag after administration of the dosage form and before the release of the pharmaceutically active agent is initiated; and 3) pulsatile release, wherein the pharmaceutically active agent is released in an immediate release or modified release fashion, e.g. sustained or delayed, followed by a time period in which there is very little or no release, followed by yet another period of immediate or modified release and so on; one or more pulses of release can be thus obtained.

As appreciated by those of skill in the art, other delivery profiles are possible and all are considered to be within the scope of controlled release for purposes of the invention.

The practice of the invention whereby the pharmaceutical composition can be chewable yet still be controlled release entails providing the pharmaceutically active agent in a controlled release multiparticulate form. In a particular practice, the active agent is provided in the form of particles having a size such that when the dosage form is chewed by an animal the active agent (particle) will not be further comminuted to any significant degree. That is, whereas some of the coated particles may indeed be crushed by chewing, the fact that there is a multiplicity of such particles (multiparticulate form) ensures that enough will survive substantially intact to provide controlled release therapy. Hence even though conventional matrix tablets lose their controlled release behavior because of the increased surface area to volume ratio (also known as specific surface area) that results from chewing, multiparticulates of the invention—which have a high specific surface area to start with—by virtue of being controlled release, e.g. coated, lessens the probability that sufficient numbers of them will be compromised by chewing, the high initial surface area to volume ratio notwithstanding.

The multiparticulate form of the pharmaceutically active agent can be fabricated by any of a variety of conventional techniques including, without limitation: balling (also known as spherical agglomeration), spray congealing, cryopelletization. The multiparticulate form can also be prepared by melt-spray congealing (MSC) techniques wherein the pharmaceutically active agent is mixed with a waxy material, heated to the melting point of the waxy material and then sprayed out from a rotary disk atomizer or other atomizer, e.g. a spray nozzle, into a congealing chamber. The resultant multiparticulates (also referred to as microspheres when produced by this technique) can then be coated or otherwise configured to provide the controlled release functionality as discussed hereinafter. Multiparticulates ensuing can also be fabricated by spray-drying a solution containing the pharmaceutically active agent and optionally other ingredients including any rate controlling excipients. Chemical methods can also be employed to manufacture the multiparticulate form, such as representatively, microencapsulation by simple or complex coacervation, interfacial polymerization and phase separation methods. Multiparticulates from such chemical methods are often referred to in the art as microcapsules or microspheres. Other methods of generating the multiparticulate form include dry or wet granulation. In dry granulation, a powder blend containing the drug is compressed into discs and the discs are subsequently milled to obtain the granules (multiparticulates). Alternatively, the powder blend is roller compacted and the compacts are subsequently milled to obtain the multiparticulates. Generally, in wet granulation, the powder blend is wet massed using an aqueous or non-aqueous solvent. The resulting granules are optionally wet milled to obtain a uniform particle size and the granules are dried e.g. in a tray or a fluid bed dryer. The multiparticulate form of the invention can also be obtained by extrusion-spheronization processes wherein a powder blend is wet massed in a manner similar to wet granulation, then extruded through an extruder to obtain spaghetti-like strands. The strands are then placed in a spheronizer, which contains a rotating bottom plate, and which shapes the wet particle into a more or less spherical shape. Core multiparticulates can also be made by a drug-layering process. Here, inert seeds, e.g. non-pareil sugar beads or microcrystalline cellulose spheres, are sprayed with a solution or a suspension containing the pharmaceutically active agent and a binder. Alternatively, dry powder containing the pharmaceutically active agent can be applied to the seeds while simultaneously spraying the seeds with a binder solution.

The foregoing methods of fabricating pharmaceutically active agent in multiparticle from are representative only, and other techniques and modifications to the above, as appreciated to the artisan, may also be employed.

In a preferred practice, the controlled release microparticulate form has an average particle size of up to about 5000 μm; more preferred is an average particle size of about 10 μm to about 5000 μm; still more preferred is an average particle size of about 50 μm to about 2000 μm; yet still more preferred is an average particle size of about 100 μm to about 1000 μm. As appreciated by the artisan, the particles for purposes of the invention can be of diverse size and shape. Also as appreciated by the artisan, the methodologies for fabricating multiparticulates as exemplified above can make the appropriate particle size in the first instance by routine adjustment of operating conditions and/or use of appropriate sizers, such as mesh screens and the like. Average particle size as referred to herein is generally connotes the mean diameter of spherical particles. As appreciated by the artisan, for shapes other than spherical, two or three dimensions may have to be specified. For example, one commonly defines an equivalent spherical diameter (i.e. the diameter of a sphere having the same volume as the particle (dv) or diameter of a sphere with the same superficial surface area as the particle (ds). Mean diameter in the current context refers generally to the mode, or the most commonly occurring value, in the particle size distribution.

Without limitation, the methodologies employable by the present invention for purposes of measuring particle sizes and particle size distributions include: image analysis (e.g. optical microscopy, electron microscopy, transmission electron microscopy); sieving (e.g. standard calibrated sieves, air-jet sieving, sonic sifters and the like); fluid classification; sedimentation methods; Coalter principle; laser methods including low angle laser light scattering methods. In a preferred practice, sieves (screens, meshes) are used; in a more preferred practice, multiple methods are used. Various particles size measurement strategies suitable for the present invention are found in the text Particle Size Measurement, Volume I, 5^{th} Edition, Terence, Allan, Chapman and Hall, 1997, the entire contents of which are incorporated herein by reference.

The mechanism of controlled release is preferably obtained by conventional routes for such drug delivery; for example and without limitation, coating the particles with materials and/or using physical configurations known in the art for the purpose of providing sustained, delayed, pulsatile or other release delivery profiles for pharmaceuticals. See generally in this regard “Multiparticulate Oral Drug Delivery” edited by Issac Ghebre-Sellassie, Marcel Dekker, Inc. 1994.

The use of coatings is preferred. Coating formulations can be either a suspension or a solution using either aqueous or organic solvents or mixtures. Coating formulations typically contain the coating polymer, one or more plasticizers, and other formulation aids such as, without limitation, detackifiers, defoamers, surfactants and the like.

For delayed release coatings, the polymer(s) used are pH sensitive, typically insoluble at low pH, e.g. pH of from 1 to about 5 as generally found in the stomach, but soluble at higher pH, e.g. greater than pH of 5.5, as typically encountered in the small intestine. Serviceable polymers for delayed release coatings include without limitation: cellulose acetate phthalate, hydroxypropylmethyl cellulose phthalte, Eudragit L100-55, Eudragit S100 and mixtures of Eudragit L100-55/S100.

For sustained release coatings, useable polymers include without limitation: hydroxypropylmethyl cellulose, ethylcellulose, Eudragit RL100, Eudragit RS100, mixtures of Eudragit RL100/RS100, Eudragit S100, Eudragit NE30D, cellulose acetate, cellulose acetate butyrate, silicone, ethylcellulose dispersions (commercially available as Aquacoat® FMC and Surrelease® (coloron).

The thickness of the coating in all events is that which is sufficient to yield necessary mechanical stability and adequate dissolution performance. While determination of appropriate thickness is thus within the skill of the art, it is preferred that for delayed release coatings, thickness be from about 20 μm to about 30 μm. For sustained release coating, preferred thickness is about 5 μm to about 50 μm.

As will be understood by the artisan, various known techniques can be used to coat the pharmaceutically active agent in multiparticulate form. By way of exemplification only, such techniques include the use of aqueous and solvent based coating systems, i.e. mixed water and organic solvents, and the use of solutions or suspensions such as latex dispersions comprised of the coating polymers. The multiparticulates can also be coated by fluidized bed equipment including top spray, rotary fluidized bed, and bottom spray beds with e.g. Wurster inserts. The multiparticles can also be coated in with side vented pan coaters typically used for coating tablets.

The amount of coating depends upon the final release profile desired and determination of same is within the ambit of routine skill. Without restriction, the coating is preferably present, in terms of weight (w/w core particles), in an amount of about 5% to about 100% by weight of the pharmaceutical composition, more preferably about 5% to 50%; still more preferably about 10% to about 50%.

The pharmaceutical composition of the invention is preferably provisioned as a dosage form whose size and shape are suitable for poke down administration; more preferably the dosage form has thereon means for enabling the division of it into smaller sizes for lesser doses, e.g. scoring and the like.

As used herein, the term “companion animal” refers to domesticated animals. Companion animals exclude humans. Preferably, the animal is a mammal. Examples of companion animals include, but are not limited to, dogs, cats and horses. The preferred companions animals are dogs and cats.

The term “palatability” means the voluntary (free choice) acceptance or ingestion of a pharmaceutical composition by companion animals, as measured by a standard palatability test, such as acceptance testing, preference testing or consumption testing. These tests are described in U.S. Ser. No. 10/091,202, filed Mar. 5, 2002, incorporated herein, supra.

The term “palatability improving agent”, as used herein, includes any composition that alters the palatability of the pharmaceutically active agent to which it is added, and more particularly improves the palatability of the pharmaceutically active agent as measured by a standard palatability test, such as acceptance testing, preference testing or consumption testing. Preferably, the difference between the voluntary acceptance rate of the pharmaceutical composition containing the palatability improving agent and the pharmaceutically active agent without the palatability improving agent is statistically significant at the 95% confidence level. Preferably, a palatability improving agent provides a voluntary acceptance by the companion animal of the pharmaceutically active agent which is greater than or equal to about 80% voluntary acceptance, and more preferably, about 90% voluntary acceptance as determined by the above mentioned tests.

“Acceptance” or “voluntary acceptance” means that the dosage form is voluntarily taken into the mouth of the animal. It is preferred that the animal voluntarily take the dosage form within its mouth within 10 minutes. It is more preferred that the animal voluntarily take the dosage form within its mouth within 5 minutes. Most preferred is that the animal voluntarily takes the dosage form within its mouth within 2 minutes.

“Pillable” means that the dosage form can be administered in the conventional manner by which tablets are given to companion animals so that the tablet is swallowed in a substantially intact form. This is also known as the “poke down” method.

“Friability” is a measure of tablet robustness to mechanical force. A standard tablet friability test is given in the United States Pharmacopea, 24^{th} edition, <1216>, Tablet Friability.

“Tablet hardness” is the force required for breaking or crushing a tablet in diametrical compression test. The test consists of placing a tablet between two anvils and applying pressure to the anvils until the tablet breaks. The force is generally measured in the units of kilopound, Newton, strong cobb or pound. In addition to being palatable, it is preferred that the dosage form should be such that is can be dosed in the conventional manner (also known as “poke down”) that is characteristic of a pillable dosage form. This is an important requirement for dosage forms that may need to be administered to animals that are too sick to accept the medication in a free choice manner or for certain animals that for some reason do not accept the dosage form by free choice on some occasions. Pillable dosage forms can be crushed or ground by the owner, caregiver, pharmacist, veterinarian so that it can be sprinkled on or mixed with food, dissolved or suspended in liquid, mixed with semisolid food products such as peanut butter or malt hairball remedies which can be administered directly or smeared onto the fur (i.e. back of a front paw) for ingestion by the animal during self grooming.

In addition to being palatable, it is preferred that the dosage form should be such that it can be dosed in the conventional manner (also known as “poke-down”) that is characteristic of a pillable dosage form. This is an important requirement for dosage forms that may need to be administered to animals that are too sick to accept the medication in a free choice manner or for certain animals that for some reason do not accept the dosage form by free choice on some occasions. Pillable dosage forms can be crushed or ground by the owner, caregiver, pharmacist, veterinarian so that it can be sprinkled on or mixed with food, dissolved or suspended in liquid, mixed with semisolid food products such as peanut butter or malt hairball remedies which can be administered directly or smeared onto the fur (i.e., back of a front paw) for ingestion by the animal during self grooming. The addition of the palatability improving agent to the pharmaceutically active agent enhances or improves the palatability of the pharmaceutical composition by improving the acceptability, such as by taste or smell, of the pharmaceutically active agent, through the introduction of a highly pronounced and desirable agent, which is attractive to the animal. Thus, if the pharmaceutically active agent is unacceptable to an animal, such as when is has a bitter taste, or alternatively, when it has a neutral taste, the palatability improving agent no only masks the undesirable flavor associated with the pharmaceutically active agent but also attracts the animal to the pharmaceutically active agent so that it voluntarily ingests the pharmaceutical composition, resulting in a palatable pharmaceutical composition. By “unacceptable” is meant bitter or neutral tasting to a companion animal such as a dog, cat or horse.

The palatability improving agents of the invention can be meat-based or non-meat based derived from meat. The term “meat” means beef, lamb, or poultry. In addition, it can be fish-based or derived from fish. The palatability improving agents are preferably non-meat based or non-meat based derived, and non-fish based or non-fish based derived. The palatability improving agents utilized in the present invention to be mixed or admixed with the pharmaceutically active agents are typically commercially available and generally acceptable for use in food applications.

The palatability improving agents of the present invention, include, but are not limited to, for example, dairy-based flavoring agents, a mixture of a natural herbs and spices, artificial egg flavor, artificial meat flavor, artificial chicken flavor, artificial fish flavor, or yeast flavor, or a combination thereof. These are commercially available.

The dairy-based flavoring agents are those derived from milk or cheese but preferably low-fat cheeses and milk, e.g. evaporated milk or skim milk or malted milk, whey or other milk products. Alternatively, the flavoring agent may be an imitation cheese (sodium capstan). Further soy or vegetable-based cheese substance may be used as the flavoring agent.

The palatability improving agents may be a mixture of natural herb and spices in combination. These natural herbs and spices include, for example, such spices as allspice, anise seed, caraway seed, cardamom, celery seed, cinnamon, cassia, clover, coriander, cumin seed, paprika, dill seed, fennel seed, ginger, mustard seed, nutmeg, saffron, black pepper, white pepper, and the like, herbs, such as basil, bay, dilled, marjoram, oregano, rosemary, sage, savory, tarragon, turmeric and thyme.

Moreover, the palatability improving agent may include seasonings, which are dry mix products containing spices and/or herbs as well as optional additional flavoring agents, salt, sugar, and starches.

The palatability improving agent may additionally be an artificial flavoring. The term “artificial” means not derived from natural animal sources. These include the fruit flavors, vegetable flavors, cheese flavors, nut flavors and the like. Many of these artificial flavors are listed in the Kirk-Othmer Encyclopedia of Chemical Technology, Vol. 11, pp. 24-28 (1994), the contents of which are incorporated by reference.

Other palatability improving agents include artificial meat, poultry, and fish flavoring agents. These include, for example, such products as artificial beef or vegetarian beef, artificial or vegetarian pork products, including vegetarian ham, vegetarian bacon, vegetarian sausage, artificial poultry (i.e. turkey, chicken and the like) products, artificial fish products, and the like. In addition, the palatability improving agent may be derived from yeast. Yeast from the group asomycetous or asporagenous may be utilitzed. Also included are the yeast like genera which belong to the order, Ustilaginales (in the Basidiomycetes) and the yeast like genera which belong to the family Sporobolomycetes and Sporobolomycetaceae. However, it is preferred that the yeast are commercially available dried yeast, such as a primary dried yeast, i.e. Saccharomyces cerevisiae, primary dried torula yeast, i.e. Torulopsis utilis and secondary yeast, i.e. brewer's dried yeast, i.e. Saccharomvces cerevisiae, and Saccharomyces carlsbergensis. In addition, the palatability improving agent may be derived from a plant source, i.e. soy meal or cotton seed oil.

The palatability improving agents utilized in the present invention are non-toxic and are food acceptable. They are preferably digestible and do not have any adverse gastrointestinal side effects associated therewith, e.g. excess flatulence or gastrointestinal pains, and the like. Moreover, the palatability improving agent is one that does not significantly affect the efficacy of the pharmaceutical active ingredient with which it is associated, i.e. it does not inhibit significantly and more preferably does not inhibit the action of the drug.

Preferred palatability improving agents include hydrolyzed vegetable protein, blends of natural flavoring and spices such as Sirius Stuff™ and Dog Bone marinade®, manufactured by Dirigo Corp., vegetarian beef, vegetarian bacon, and roast garlic, manufactured by Geneva Ingredients, Inc., blends of dried skim milk, malted milk, whey and other products, such as All diary Blend™, yeast flavoring, especially 100% Saccharomyces cerevisiae, such as Brewtech™ Dried Brewer's Yeast, blends of animal proteins and fat formulated to replace whole egg, such as Eggsact™, and blends of white and yellow cheese product powders, and cheese rind such as Cheese Plus Cheese™, manufactured by International Ingredients Corp., peanut butter and artificial chicken, manufactured by Bush Bake Allan Americas, artificial beef manufactured by Pharmachemie at Syracuse, Nebr., or mixtures thereof.

The palatability improving agent is present in the palatable pharmaceutical composition in amounts effective to make the pharmaceutical palatable to the companion animal and if the pharmaceutical has an unacceptable flavor, in amount effective to mask the off flavor, i.e. palatability improving amounts. It is preferred that the palatability improving agent can be present in amounts ranging from about 0.025% to about 99% by weight of the pharmaceutical dosage form, more preferably the palatability improving agent is present in the amount ranging from about 0.75% to about 50% and most preferably from about 1% to about 25% by weight of the palatable pharmaceutical composition; in both the foregoing instances, yeast is preferably excluded from these percentage limitations. With respect to the yeast flavoring, it is preferred that the yeast be present in amount ranging from about 2% to about 25% by weight of the pharmaceutical compositions, more preferably from about 5% to about 20% by weight of the pharmaceutical composition.

The palatability improving agent is given to the companion animal in association with pharmaceutically active agents, e.g., veterinarian drugs, normally given to companion animals including without limitation: amebicides, trichomonacides, analgesics, anorexics, antiarthritics, antibacterials, antibiotics, anticoagulants, antidepressants, antihistamines, antineoplastics, anti-Parkinsonism, drugs, antipyretics, anti-spasmodics, anticholinergics, antiviral agents, cardiovascular drugs, contraceptives, diuretics, fertility agents, hemantinics, hormones, laxatives, parasympathetic agents, parasympathomometics, psychostimulants, sedatives, sympathomimetics, anti-inflammatory agents, barbiturates, stimulants, tranquilizers, and the like. Examples include carprofen, selegeline, icopexil, methamphetamine, methcyclothiazide, cephalexmin, cephaloglycin, cloxacillin, phenoxyethyl penicillin, erythromycin, pargyline, ephedrine, codeine, methycyclothiazide, metharbital, deserpidine, pentobarbital, isoproterenol, peperazine, estrone, hydrochlorothiazide, ethchlorvynol, chlorazepate, sulfamethizole, phenazopyridine, oxytetracycline, pentaerythritol tetranitrate, diethylstilbestrol, 1-hyoscyamine, ethaverine, pentylenetetrazol, griseofulvin, ampicillin, phendimetrazine, meprobamate, conjugated estrogens, testosterone, pralidoxime, dicloxacillin, isoniacid, methanamine mandelate, phenacetain, aspirin, caffeine, hydrocodone bitartrate, oxacillin, phentermine, bisacodyl NF, phenmetrazine, ephedrine, glyceryl guaiacolate, phenobarbital, theophylline, sulfonamide, phenoxymethyl penicillin, kanamycin, tetracycline, hetacillin, metampicillin, aluminum glycinate, acetaminophen, salicylamide, methyltestosterone, bephenium hydroxynaphthoate, erythrityl tetranitrate, procyclidine, digoxin, cyclizine trimethoprim, sulfamethoxazole, benzyl penicillin, papaverine, hydralazine, allobarbital, acetaminophen, methandrostenolone, dimethindene, xylometazoline, tolazoline, tripenalennamine, reserpine, adiphenine, ethinamate, belladonna, piperacetazine, rifampin, warfarin, promethazine, sulfinpyrazone, phenylbutazone, oxyphenbutazone, carbamazepine, imipramine, furosemide, glycerol trinitrate, isoproterenol, bromisovalum, pentylenetetrazol, isometheptene, oxyphenonium bromide, amantadine, lithium carbonate, butyrophenone, hydroxyzines, chorionic gonadotropin, menotropins, cyanocobalamin, dipyridamole, casanthranol, dioctyl sodium sulfosuccinate, methylphenidate, thyroxine, amphetamine, chlordiazepoxide, diazepam and sulfisoxazole, Cephalexin; Chloramphenicol; Lincomycin; Lincomycin hydrochloride monohydrate; Oxytetracycline; Tetracycline; Tylosin, Salicylazosulfapyridine (“Azulfidine”); Sulfadimethoxine; Trimethorprim-sulfadiazine (“Tribrisssen”), Corticotropin (ACTH); Cortisone acetate; Deoxycorticosterone acetate (DOCA); Dexamethoasone; Hydrocortisone acetate; Phenylbutazone; Prednisolone, Mibolerone; Progesterone; L-Thyroxin (T₄, tetraiodothyronine), Aracoline acetarsol; Arecoline hydrobromide; Bephenium embonate (or hydroxynaphthoate); Bunamidine hydrochloride; Diethylcarbamazine citrate; Dichlorophen; Disophenol; Hexylresorcinol; Mebendazole; Niclosamide; Piperazine salts, Barbituric acid, Phenobarbital sodium, thiopental sodium, Amphetamin, dextroamphetamine, Diphenylhydantoin, Phenobarbital, Acepromazine maleate; Chlorpromazine; Meperidine hydrochloride; Meprobamate, Norpinephrin, epinephrine, isoproternol, ephedrine, atropine, methscopolamine, Chlorpheniramine maleate; Tripelennamine, Amphetamine sulfate; Bethanechol chloride; Cyclophosphamide; Mitotane (o,p' DDD); D-Penicillamine, Mercaptomerin, chlormerodrin, acetazolamide, cyclothiazide, chlorothiazide, Meperidine, Darbazine, Digoxin, quinidine, procainamide, lidocaine, aminophylline, and the like.

In general, any improvement in acceptance of a palatable dosage form containing pharmaceutically active ingredients is desirable over pharmaceutical dosage forms that are not formulated to increase palatability. It is preferred that the palatable dosage form have an acceptance rate of about 30% or greater. More preferred is a palatable dosage form with an acceptance rate of about 50% or greater. Even more preferred is a palatable dosage form with an acceptance rate of about 80% or greater. Most preferred is a palatable dosage form with an acceptance rate of about 90% or greater.

The pharmaceutically active agent is present in amounts effective to treat a particular disease or in prophylactically effective amounts. The pharmaceutically effective amount varies with each drug and is determined by the veterinarian prescribing the drug.

The veterinarian will determine the dosage of the present pharmaceutically active agents which will be most suitable. The amount will depend upon several factors. For example, it will vary with the form of administration and the particular compound chosen, and furthermore, it will vary with the animal under treatment, the age of the animal, the weight of the animal and the type of malady being treated. However, the effective amount of drug to be delivered would be no different if palatability improving agent were not present.

The palatable pharmaceutical composition may be orally administered, for example, with an inert diluent or with an assimilable edible carrier, or it may be enclosed in hard or soft shell gelatin capsules, or it may be compressed into tablets, or in the form of troches, or it may be incorporated directly into the food of the diet. For oral therapeutic administration, the active compound, and the flavoring agent may be incorporated with excipients and used in the form of ingestible tablets, troches, capsules and wafers, or alternatively, can be administered in liquid form.

The palatability improving agent can be added as a coating to the dosage form or either included in or separate from the controlled release coatings. Alternatively, the palatability improving agent can be sprayed onto the surface of a table or pill containing the pharmaceutical agent. The pharmaceutical composition may contain an anti-mycotic and/or anti-bacterial agent. Preferably, the palatability improving agent increases or at least does not decrease the shelf life of the pharmaceutically active agent. Furthermore, the palatability improving agent enhances compliance with a therapeutic program for companion animals.

The tablets, troches, pills, capsules and the like may also contain the following: a binder such as sodium starch glycosate, gum tragacanth, acacia, polyvinylpyrrolidone, corn starch or gelatin; excipients such as dicalcium phosphate, and microcrystalline cellulose; a disintegrating agent such as corn starch, potato starch, alginic acid, and the like; a lubricant such as magnesium stearate, stearic acid, polyethylene glycol, talc or silica. When the dosage unit form is a capsule, it may contain, in addition to materials of the above type, a liquid carrier.

Coatings or other components may me present so as to modify the physical form of the pharmaceutical composition. For instance, tablets, pills, or capsules may be coated with shellac, sugar or both, sweetening agent, methyl and propylparabens as preservatives, coloring agents, a dye and other ingredients such as cherry. The palatable pharmaceutical composition is preferably prepared in unit dosage form. In such form, the preparation is subdivided into unit doses containing appropriate quantities of the pharmaceutically active agent in association with the palatability improving agent. The unit dosage form can be in packaged preparation, such as packaged tablets, capsules, pills, lozenges, troche and the like. The preferred solid unit-dosage form is a hard, compressed tablet. Of course, any material used in preparing any dosage unit form should be pharmaceutically pure and substantially non-toxic in the amounts employed.

As used herein, “pharmaceutically acceptable carrier” includes any and all solvents and dispersion media for pharmaceutically active substances are well known in the art. Except insofar as any conventional media or agent is incompatible with the active ingredient, its use in the therapeutic compositions of the present invention is contemplated. More than one active ingredient can also be incorporated into the palatable pharmaceutical compositions.

The preparation of the palatable pharmaceutical compositions of the present invention can be accomplished by utilizing any one of a wide variety of different known methods. One such method is by wet granulation in which the components, e.g., pharmaceutically active agent, the palatability improving agent, and excipients are mixed with a wet granulating solvent, such as an aqueous or a non-aqueous solvent or solvent mixtures, e.g., as water or alcohol, in a mixing apparatus. The mixture is dried using techniques known in the art and then the dried blend is generally processed further by sizing the granulation through a mill to reduce the size of the particles. Lubricant and any additional excipients are then added and blended to provide a uniform homogeneous mixture. In another variation, the blend is simultaneously granulated in the granulating vehicle and dried using a fluid bed granulation process. The resulting granules are milled and then blended with a lubricating agent.

The palatable pharmaceutical compositions of the present invention may be prepared by dry formulation in which the pharmaceutically active agent, the palatability improving agent and the carrier material are thoroughly intermixed. Excipients, binding agents, lubricants, disintegrating agents, and colorants, if necessary are homogeneously mixed. Examples of suitable excipients are lactose and sodium starch glycolate.

The resulting blend is then made into solid dosage form. If the final dosage form is a tablet or chewable tablet, the composition is transferred to a tablet press and compressed into a tablet at an appropriate compression pressure to achieve, preferably, a hardness in the range of from about 5 to about 25 kP, at compression pressures of about 1600 to about 2000 pounds/square inch. The product thus obtained has the desired hardness, and low level of friability found in tablets.

Alternatively, the blend may be encapsulated by a gelatin shell to form a capsule utilizing techniques known to one of ordinary skill in the art. Similarly, pills and troches are prepared using conventional techniques known to the skilled artisan.

Alternatively, the palatable pharmaceutical compositions of the present invention may be formed into shapes, textures, and mimic structures so as to simulate foods, such as biscuits, cheeses, meat scraps and the like.

The present inventors have found that when the palatability improving agents of the present invention are added to pharmaceutically active agents, the companion animals were not only attracted thereto, but also freely ingested and swallowed the pharmaceutical compositions containing the palatability improving agents. These are described in the following examples.

Moreover, the present inventors have found that the addition of the palatability improving agents in palatable enhancing effective amounts made the pharmaceutical compositions more stable and increased shelf-life when the palatability improving agent of the present invention was present. Unless indicated to the contrary, the percentages are by weight of the pharmaceutically composition.

The term “effective” amount of a drug is meant a non-toxic but sufficient amount of compound to provide the desired therapeutic or prophylactic effect.

“Carriers” or “vehicles” as used herein refer to carrier material suitable for solid oral drug administration and include any such materials known in the art, e.g., diluent, binders, granulating agents, disintegrates, lubricating agents, colorants and the like.

The flavoring agents used in the Examples are as follows: Vegetarian beef flavor, Geneva Ingredients, Inc, Waunakee, Wis., is a mixture of maltodextrin, autolyzed yeast extract, natural flavors, partially hydrogenated vegetable oil (soybean and/or cottonseed), onion powder, and silicon dioxide. Vegetarian bacon flavor, Geneva Ingredients, Inc., Waunakee, Wis., is a mixture of maltodextrin, natural flavors, peanut oil, natural smoke flavor, and silicon dioxide.

Roast garlic flavor, Geneva Ingredients, Inc., Waunakee, Wis., is a mixture of salt, maltodextrin, autolyzed yeast extract, natural flavors, partially hydrogenated vegetable oil (cottonseed or soybean) and silicon dioxide.

Artificial powdered beef flavor, Pharma Chemie, Lincoln, Nebr., is a mixture of hydrolyzed vegetable protein, natural flavor, and hydrogenated vegetable oils.

Brewtech(Dried Brewers Yeast, International Ingredient Corporation, St. Louis, Mo., is 100% dried Saccharomyces cerevisiae from the brewing industry that is distilled to remove the alcohol, naturally debittered, and roller dried.

Eggsact(, Dried egg replacer, International Ingredient Corporation, St. Louis, Mo., is a special blend of animal proteins and fat formulated to replace or extend whole eggs. Cheese Plus Cheese product, International Ingredient Corporation, St. Louis, Mo., is a blend of white and yellow cheese product powders, and cheese rind.

Sugar foods by-product, International Ingredient Corporation, St. Louis, Mo., is produced from the by-products of dry packaged drink mixes, dried gelatin mixes, hard candy, and similar specialty food products that have a high sugar content, and citric acid.

Trusil N/A Peanut flavor, Bush Boake Allen Americas, Chicago, Ill., is a trade-secret mixture of flavor items on the FEMA/GRAS list.

Artificial chicken flavor, Bush Boake Allen Americas, Chicago, Ill., is a trade-secret mixture of flavor items on the FEMA/GRAS list.

Sirius Stuff™, Dirigo Corporation, Boston, Mass., is a blend of yeast, garlic, salt, herbs, kelp and fermented soy.

The following non-limiting Examples further illustrate the invention.

### EXAMPLES

Definitions

Three different hydrophilic polymers were used to formulate controlled release matrix tablets as indicated later herein. These are:

1) Methocel® polymers (Dow Chemical Company, Midland, Mich.) are hydroxypropyl methylcellulose (HPMC) polymers. They are available in USP, JP, EP grades. There are multiple polymer grades available, as shown in Table A, which represent a variety of viscosities and hydration rates.

TABLE A
USP Grades of Methocel ® (Premium Products)

| Methocel | | | | | | |
|---|---|---|---|---|---|---|
| Premium | | | | | | E |
| Product | K | K | K | K | E | 10 MP |
| Grade* | 100 LVP | 4 MP | 15 MP | 100 MP | 4 MP | (CR Only) |
| USP | 2208 | 2208 | 2208 | 2208 | 2910 | 2910 |
| Substi- | | | | | | |
| tution | | | | | | |
| Type** | | | | | | |
| Nominal | 100 | 4000 | 15000 | 100000 | 4000 | 10000 |
| Viscosity, | | | | | | |
| 2.0% in | | | | | | |
| water | | | | | | |
| Methoxyl | 19-24 | 19-24 | 19-24 | 19-24 | 28-30 | 28-30 |
| (%) | | | | | | |
| Hydroxy- | 7-12 | 7-12 | 7-12 | 7-12 | 7-12 | 7-12 |
| propoxyl | | | | | | |
| (%) | | | | | | |
| Moisture | 3 | 3 | 3 | 3 | 3 | 3 |
| (%) as | | | | | | |
| packaged | | | | | | |
| (max.) | | | | | | |

*Also available in faster hydrating controlled release (CR) grades
**Also available in EP and JP grades
Reference: Dow Chemical, “Formulating for CR with Methanol Premium cellulose esters”, 1995; incorporated herein by reference.

Hydration of the HPMC polymer leads to gel formation at the surface and consequently slows water penetration into the tablet core. The faster the polymer hydration rate, the more likely adequate sustained release properties will be observed due to rapid initial gel layer formation. The gel strength is a direct function of both polymer viscosity and concentration. For Methanol® Premium products, the “K” series is the fastest to hydrate (followed by the Methocel® E Premium products). This is due to the combined effects of a lower substitution level for hydrophobic methoxyl group and a higher level of the hydrophilic hydroxypropoxyl substitution.

The viscosity of Methocel® gels is relatively pH independent; however, if drug solubility varies dramatically over a given pH range, the release may be pH dependent.

Methocel® products are free-flowing white to off-white powders which are available in 50 lb. multiwall bags and have a shelf life of 36 months. For Methocel® premium grades, the particle size is 100%<30 mesh (99%<40 mesh). Controlled release grades are available which are faster hydrating and have a smaller similar particle size (E series 95%<100 mesh, K series 90%<100 mesh).

2) Polyox (Water Soluble Resins (WSR) (The Dow Chemical Company, Midland, Mich., formerly Union Carbide Corp.) are nonionic poly(ethylene oxide) polymers. They are available in a large range of molecular weights as shown in Table B.

TABLE B
NF Grades of Polyox (Water-Soluble Resins)

| Polyox (NF Grade) | Approximate Molecular Weight |
|---|---|
| WSR N-10 | 100,000 |
| WSR N-8OL | 200,000 |
| WSR N-80H | 200,000 |
| WSR N-750 | 300,000 |
| WSR N-3000 | 400,000 |
| WSR-205 | 600,000 |
| WSR-1105 | 900,000 |
| WSR N-12K | 1,000,000 |
| WSR N-60K | 2,000,000 |
| WSR-301 | 4,000,000 |
| WSR Coagulant | 5,000,000 |
| WSR-303 | 7,000,000 |

When used in a conventional matrix tablet, Polyox hydrates rapidly to form a gel layer on a tablet surface. Release of the pharmaceutically active agent proceeds by diffusion through this gel layer and subsequent tablet erosion. Since Polyox® polymers are nonionic, no interaction is expected with active drug substances.

Storage stability can be an issue for tablets composed of polyethylene oxide due to the potential for chain cleavage via autooxidation. E.g.:

Butylated hydroxytoluene (BHT) and Vitamin E efficiently stabilize Polyox (WSR under storage and use condition.

Product stability is greatly improved by minimizing long term exposure of the polymer high temperature and oxygen.

Tablets can be effectively stabilized by controlling the antioxidant concentration in the final formulation.

All Polyox® (WSR grades are supplied with 100 to 1000 ppm BHT for antioxidant purposes.

3) Carbopol® resins (Noveon, Inc., Cleveland, Ohio).

Carbopol® resins are very high molecular weight polymers of acrylic acid, which are chemically crosslinked with polyaklenyl alcohols or divinyl glycol. Carbopol® (resins to not dissolve in water but rather form colloidal gel dispersions. Carbopol® resins are available in three grades: 934P NF, 971P NF, and 974P NF. Grades are differentiated based on degree of crosslinking, crosslinker, and polymerizing solvent as described in Table C. 971P and 974P are the preferred grades due to the presence of low level benzene residuals in 934P.

TABLE C
NF Grades of Carbopol ® resins

| | Carbopol | | |
|---|---|---|---|
| Polymer | 934P NF | Carbopol 971P NF | Carbopol 974P NF |
| Crosslinker | Allyl sucrose | Allyl pentaerythritol | Allyl pentaerythritol |
| Polymerizing | Benzene | Ethyl acetate | Ethyl acetate |
| Solvent | | | |
| Degree of | High | Low | High |
| Crosslinking | | | |

Although Carbopol® resins are not water soluble, they are hydrophilic and absorb water readily. Mechanism of release from Carbopol® matrix tablets is conceptually different from matrix tablets comprised of water soluble polymers. Upon hydration, Carbopol® quickly swells to form a gel at the surface interface. When fully hydrated, osmotic pressure from within works to break up the structure, essentially by sloughing off discrete pieces of the hydrogel. These hydrogel pieces remain intact, and the drug releases by diffusion through the gel layer.

The swelling of Carbopol® resins is affected by the pH of the surrounding media. Peak swelling is seen in the range of pH 5-9. This leads to pH dependent drug release from Carbopol® matrix tablets.

Carbopol® resins are synthetic polymers, which tend to be more consistent than semisynthetic or natural products. Storage stability testing performed by BF Goodrich suggests chemical stability. For example, Carbopol® 934P has been kept at room temperature and 80° C. for two years, and test theophyline tablets were made monthly. No significant changes were observed in the release profiles.

Other materials used in the following examples are described below:

a) Aquacoat® (FMC Corporation, Philadelphia, Pa.) is an aqueous dispersion (total solids approximately 30%) of the polymer ethylcellulose in water. It also contains small amounts of sodium lauryl sulfate and cetyl alcohol. When sprayed onto a surface, the dispersion medium (water) evaporates, and the individual, sub-micron sized polymer particles coalesce to form a film. This film provides a diffusion barrier for the drug molecules which provides sustained release of the drug over a prolonged duration.

b) Eudragit S100 (Rohm Pharma, Piscataway, N.J.) is an anionic copolymer made from methacrylic acid and methacrylate. It is insoluble in acids but becomes soluble in intestinal fluid from pH 7 upwards.

### Example 1

This example shows the increase in voluntary acceptance (free choice) by dogs of placebo tablets having a palatability improving agent therein (flavored) to tablets having no such agent (unflavored) or having Bitrex (having a known offensive and bitter taste).

This example shows that dogs accept flavored tablets more readily than tablets that do not contain a flavor or tablets that contain a known unpleasant tasting material such as Bitrex.

A cohort of 25 dogs was tested, each dog was offered the choice of three of five treatments. The dogs were fasted overnight and were offered the tablets in their usual food dishes. The dishes were removed after 5 minutes. The placebo with Bitrex was evaluated in a separate but similar study. The testing results are shown in Table 1.

TABLE 1
Canine acceptance of unflavored and flavored placebo tablets.

| Formulation | % Free Choice Acceptance Rate |
|---|---|
| Unflavored placebo | 68% |
| Placebo with Bitrex | 44% |
| 1% Artificial Beef Flavor | 92% |
| 5% Artificial Beef Flavor | 96% |
| 10% Artificial Beef Flavor | 96% |
| 1% Brewer Yeast Flavor | 79% |
| 10% Brewers Yeast Flavor | 91% |

### Example 2

This example shows the controlled release properties (dissolution over time) of matrix tablets containing carprofen made by prior art methods using various polymer coatings.

Prototype controlled release matrix tablets containing carprofen were made by a direct compression process using the Manesty Type F tablet press (Manesty, Knowsley, Merseyside, United Kingdom). The tablets contained a polymeric excipient, which moderated the release of carprofen. Carprofen, lactose fast-flo, and the polymer were blended together for 20 minutes. The blend was then passed through a #40 mesh screen and blended for an additional 20 minutes. Magnesium stearate (1% of the total blend weight) was added and blended for an additional 3 minutes. For the smaller 25 mg tablets, 0.4″×0.2″ tooling was used and for the larger 100 mg tablets, 0.635″×0.3175″ caplet shaped tooling was used. Depending on the formulation, tablet hardness of 112 Kp to 17 Kp were achieved. A summary of the manufactured lots is given in Table 2. Average dissolution profiles (in vitro, given in percent as a function of time at pH of 7.5 for these formulations at different coating polymer levels and types) is at Table 3.

TABLE 2
Summary of controlled release matrix tablets
manufactured for screening.

| | | Tablet Weight | |
|---|---|---|---|
| Lot Number | Potency (mg) | (mg) | Polymer level/type |
| 37255-008 | 100 | 600 | 30% Methocel K4M |
| 37255-009 | 100 | 600 | 30% Methocel K100LV |
| 37255-010 | 100 | 600 | 30% Carbopol 971P |
| 37255-011 | 100 | 600 | 20% Polyox Coagulant |
| 37255-012 | 100 | 600 | 30% Polyox N-750 |
| 37255-028 | 25 | 150 | 30% Methocel K4M |
| 37255-029A | 25 | 150 | 10% Carbopol 971P |
| 37255-029B | 100 | 600 | 10% Carbopol 971P |
| 37255-030A | 25 | 150 | 15% Polyox Coagulant |
| 37255-030B | 100 | 600 | 15% Polyox Coagulant |
| 37255-040 | 100 | 600 | 30% Methocel K4M |
| 37255-041 | 100 | 600 | 15% Polyox Coagulant |
| 37255-045A | 25 | 600 | 30% Methocel K4M |
| 37255-045B | 25 | 600 | 25% Methocel K4M |
| 37255-046A | 25 | 600 | 15% Polyox Coagulant |
| 37255-046B | 25 | 600 | 10% Polyox Coagulant |

TABLE 3
Average dissolution (%) profiles at pH 7.5 for formulations in Example 2.

| Lot Number | 0.5 hr | 1.0 hr | 2.0 hrs | 4.0 hrs | 8.0 hrs | 12.0 hrs | 16.0 hrs | 20.0 hrs | 24.0 hrs |
|---|---|---|---|---|---|---|---|---|---|
| 37255-008 | 7.9 | 11.4 | 19.5 | 34.4 | 60.8 | 74.2 | 82.1 | 86.7 | 89.5 |
| 37255-009 | 14.2 | 23.1 | 62.0 | 85.8 | 93.7 | 95.1 | 94.8 | 95.6 | 95.6 |
| 37255-010 | 0.4 | 0.7 | 1.7 | 4.3 | 12.1 | 22.7 | 33.3 | 44.1 | 53.5 |
| 37255-011 | 3.2 | 4.8 | 7.9 | 14.0 | 27.1 | 39.8 | 52.6 | 64.3 | 75.0 |
| 37255-012 | 4.4 | 10.0 | 24.6 | 53.8 | 91.8 | 96.6 | 96.5 | 97.0 | 97.8 |
| 37255-028 | 19.7 | 33.9 | 55.8 | 78.8 | 92.4 | 93.9 | 93.7 | 94.3 | 95.4 |
| 37255-29A | 1.2 | 2.2 | 4.7 | 12.5 | 70.8 | 92.2 | 97.6 | 97.4 | 97.1 |
| 37255-29B | 0.6 | 1.1 | 2.7 | 6.2 | 14.4 | 23.1 | 33.1 | 46.5 | 92.5 |
| 37255-30A | 5.9 | 9.4 | 16.6 | 33.0 | 63.3 | 89.3 | 97.9 | 98.2 | 99.3 |
| 37255-30B | 6.5 | 8.7 | 13.5 | 21.5 | 38.0 | 49.8 | 68.2 | 81.0 | 93.7 |
| 37255-40 | 5.4 | 8.6 | 13.7 | 22.6 | 36.7 | 47.4 | 56.2 | 63.6 | 69.6 |
| 37255-41 | 4.0 | 6.0 | 9.7 | 17.7 | 34.3 | 50.0 | 61.9 | 71.6 | 80.8 |
| 37255-45A | 5.3 | 9.4 | 17.0 | 30.5 | 52.9 | 69.3 | 82.0 | 91.1 | 97.4 |
| 37255-45B | 7.0 | 13.0 | 23.9 | 40.7 | 67.1 | 84.7 | 94.3 | 99.2 | 101.4 |
| 37255-46A | 6.1 | 9.4 | 15.4 | 26.7 | 48.3 | 67.5 | 81.7 | 89.3 | 92.5 |
| 37255-46B | 10.2 | 14.4 | 21.4 | 33.7 | 56.7 | 73.3 | 82.7 | 88.1 | 88.5 |

### Example 3

This example shows the controlled release properties (dissolution over time) of matrix tablets containing carprofen using specific polyox polymers.

Formulations containing 20.08% carprofen, lactose fast-flo as filter, polymer, and 1% magnesium sterarate as the lubricant were manufactured by a direct compression process similar to that given in Example 2. The proportion of polymer and lactose fast-flo were varied. The total weight of the tablets and the tooling used was dependent on the tablet strength. The polymer levels for the three strengths were as given in Table 4. Average dissolution profiles (in vitro) in percent as a function of time at pH of 7.5 for these formulations is given at Table 5.

TABLE 4
Polyox grades and levels used in screening controlled
release carprofen matrix tablets.

| 50 mg carprofen | 150 mg carprofen | |
|---|---|---|
| (250 mg total | (750 mg total | |
| tablet weight) | tablet weight) | 200 mg carprofen (1000 |
| Tooling: | Tooling: | mg total tablet weight) |
| 0.458″ × 0.229″ | 0.635″ × 0.3175″ | Tooling: 0.727″ × 0.3635″ |
| caplet | caplet | caplet |
| Lot 36423-154A | Lot 36423-156 | Lot 36423-157B |
| 20% Polyox WSR | 30% Polyox WSR 205 | 20% Polyox WSR 205 (MW |
| 301 (MW 4M) | (MW 600K) | 600K0 |
| Lot 36423-154B | Lot 36423-157A | Lot 36423-158B |
| 30% Polyox | 30% Polyox WSR | 30% Polyox WSR N-750 |
| WSRN-60K | 1105 (MW 900K) | (MW 300K) |
| (MW 2M) | | |
| Lot 36423-154C | Lot 36423-158A | Lot 36423-159B |
| 30% Polyox WSR | 30% Polyox WSR | 30% Polyox WSR 205 (MW |
| 301 (MW 4M) | N-750 (MW 300K) | 600K) |
| Lot 36423-154D | Lot 36423-159A | |
| 15% Polyox WSR | 20% Polyox WSR 205 | |
| Coagulant | (MW 600K) | |
| (MW 5M) | | |

TABLE 5
Average dissolution (%) profiles at pH 7.5 for formulations in Example 3.

| Lot Number | 0.5 hr | 1.0 hr | 2.0 hrs | 4.0 hrs | 8.0 hrs | 12.0 hrs | 16.0 hrs | 20.0 hrs | 24.0 hrs |
|---|---|---|---|---|---|---|---|---|---|
| 36423-154A | 5.1 | 7.8 | 12.8 | 23.9 | 45.3 | 65.0 | 79.8 | 94.9 | 102.5 |
| 36423-154B | 3.4 | 5.8 | 11.5 | 24.9 | 52.6 | 76.0 | 91.2 | 107.1 | 102.7 |
| 36423-154C | 2.5 | 4.0 | 7.4 | 14.6 | 30.8 | 46.8 | 61.5 | 75.9 | 86.3 |
| 36423-154D | 9.8 | 14.3 | 19.6 | 33.3 | 60.2 | 78.7 | 94.5 | 97.5 | 98.3 |
| 36423-156 | 9.4 | 13.3 | 21.3 | 36.9 | 66.9 | 94.0 | 101.4 | 101.8 | 101.9 |
| 36423-157A | 6.6 | 11.0 | 20.8 | 42.0 | 83.6 | 100.8 | 102.4 | 102.6 | 102.9 |
| 36423-158A | 3.6 | 10.1 | 25.7 | 55.9 | 96.9 | 99.8 | 99.9 | 99.9 | 100.0 |
| 36423-158B | 4.1 | 8.7 | 18.7 | 52.0 | 92.1 | 98.3 | 98.7 | 98.9 | 99.2 |
| 36423-159A | 17.1 | 21.4 | 36.7 | 62.9 | 97.9 | 102.5 | 102.9 | 102.9 | 102.8 |
| 36423-159B | 13.4 | 20.5 | 40.3 | 62.5 | 85.4 | 98.6 | 99.7 | 99.8 | 99.9 |

### Example 4

This example reports on the in vivo performance of controlled release matrix tablets.

The example shows the in vivo performance of selected controlled release matrix tablet formulations. Compared to immediate release formulations, controlled release formulations typically show lower maximum plasma concentration (Cmax; in units of μg/ml) and longer times (Tmax; units of hr) to reach Cmax values.

The in vivo pharmacokinetic performance of selected controlled release matrix tablets containing carprofen was determined in laboratory beagle dogs in a non-cross over fashion. Although both the R- and S-carprofen concentrations were determined, only the data for total carprofen concentrations are presented here. For comparison, and as a control, dogs were administered (poke down) an immediate release (IR) carprofen dose of 2 mg/lb given as a single dose and as 2×1 mg/lb given in a twice daily (BID) fashion. The resulting pharmacokinetic parameters were calculated and are given in Table 6. The Cmax and AUC values (Area under the Plasma-time Curve; units of μg/ml-hr) are shown dose-normalized to 2 mg/lb. Values in parenthesis are standard deviations.

TABLE 6
Summary of pharmacokinetic studies with controlled
release matrix tablets.

| Lot Number | | In vitro | | | |
|---|---|---|---|---|---|
| and Dose | Formulation | release | Cmax | Tmax | AUC |
| 2 mg/lb | Immediate | | 54.7 | 1.1 | 401 |
| | Release | | (6.5) | (0.5) | (64) |
| 1 mg/lb BID | Immediate | | 28.6 | 0.8 | 412 |
| | Release | | (5.7) | (0.3) | (127) |
| 37255-040 | 30% Methocel | ˜70% in 24 hr. | 5.9 | 3.3 | 88 |
| 100 mg | K4M | | (2.6) | (1.5) | (49) |
| 37255-041 | 15% Polyox | ˜80% in 24 hr. | 12.2 | 7.5 | 190 |
| 100 mg | Coagulant | | (6.1) | (3.4) | (74) |
| 37255-029 | 10% Carbopol | ˜90% in 12 hr | 26.2 | 1.8 | 195 |
| 2 × 25 mg | 971P | | (3.7) | (0.5) | (41) |
| 37255-030 | 15% Polyox | ˜90% in 12 hr | 17.8 | 4.0 | 176 |
| 2 × 25 mg | Coagulant | | (4.6) | (2.3) | (48) |

### Example 5

This example establishes that controlled release matrix tablets containing carprofen and a palatability improving agent can be made.

This example teaches how to make flavored controlled release matrix tablets. Flavored matrix tablets with other flavors such as Brewer's yeast and Artificial Powdered Beef can be manufactured similarly. The level of flavor should be chosen such that the resulting tablets are palatable to dogs, cats, or other companion animal of interest. The type of rate controlling polymer (e.g. Methocel, Polyox or Carbopol) and its level in the tablet formulation should be chosen such that the pharmacologicallya active agent incorporated in the tablets is released at the desired in vitro release rate and has the desired in vivo performance characteristics. The total tablet weight should be such that each tablet contains the desired quantity of the active agent. The manufacturing parameters such as tablet tooling, and tablet hardness should be appropriate for the application. Other methods of producing tablets such as dry and wet granulation, including roller compaction and fluid bed granulation can be used as appropriate. These selections are obvious to one skilled in the art.

Flavored controlled release matrix tablets containing 25 mg carprofen were manufactured by a direct blend and compress process similar to the one described in Example 2. In Table 7, Formulation 37255-122A consisted on 16.7% carprofen, 60.3% lactose fast-flo, 15% Polyox Coagulant (MW 5M), 7% Sirius Stuff as the flavor ingredient, and 1% magnesium stearate. Formulation 37255-122B consisted of 16.7% carprofen, 59.8% lactose fast-flo, 15% Polyox Coagulant (MW 5M), 7.5% Cheese Plus Cheese as the flavor ingredient, and 1% magnesium stearate.

### Example 6

This example report on the in vivo performance of controlled release matrix tablets having a palatability improving agent therein.

This example shows the in vivo performance of selected flavored controlled release matrix tablet formulations. Compared to immediate release formulations, controlled release formulations typically show lower Cmax and longer Tmax values. The presence of a palatability improving agent did not change the pharmacokinetic characteristics of the matrix tablets. Note that the tablets were dosed in the conventional (poke-down)manner and not by the free-choice acceptance method.

The flavored CR matrix tablets described in Example 5 were studied in fasted beagle dogs. The in vivo performance is characteristic of a controlled release tablet with a lower Cmax and a longer Tmax. The bioavailability relative to an immediate release formulation was reduced to ˜30%. The pharmacokinetic parameters are summarized in Table 7. The comparisons are relative to the IR formulation.

TABLE 7
Summary of pharmacokinetic studies with flavored
controlled release matrix tablets.

| | | | | | AUC |
|---|---|---|---|---|---|
| | | | | | (dose |
| Lot Number | | In vitro | | | normalized |
| and Dose | Formulation | release | Cmax | Tmax | to 2 mg/lb) |
| 2 mg/lb | Immediate | | 54.7 | 1.1 | 401 |
| | Release | | (6.5) | (0.5) | (64) |
| 1 mg/lb BID | Immediate | | 28.6 | 0.8 | 412 |
| | Release | | (5.7) | (0.3) | (127) |
| 37255-122A | 15% Polyox | ˜90% in 12 hr | 13.4 | 5.0 | 118 |
| 2 × 25 mg | Coagulant | | (5.2) | (1.2) | (20) |
| flavored CR | Sirius Stuff | | | | |
| | flavor | | | | |
| 37255-122B | 15% Polyox | ˜90% in 12 hr | 13.3 | 5.5 | 110 |
| 2 × 25 mg | Coagulant + | | (4.5) | (1.0) | (44) |
| flavored CR | Cheese Plus | | | | |
| | Cheese | | | | |
| | Flavor | | | | |

### Example 7

This examples observes canine testing behavior in palatability tests of flavored drug containing tablets. In particular, this example demonstrates that dogs will chew flavored tablets before swallowing.

Palatability studies are conducted in 40 random source dogs of various breeds. Initial studies monitored the acceptance and consumption of placebo tablets, which varied in flavor and size. Favorite flavors were then re-tested in compressed tablets containing carprofen. In these studies, the acceptance and consumption behavior of the dogs was monitored, in particular, whether the tablets were chewed before swallowing.

In all instances of the dogs accepting the flavored tablet and consuming it, the study monitors noted that most dogs chewed the flavored tablets before swallowing. It was estimated that the dogs chewed the tablets more than two times. In some cases, it was estimated that the dogs chewed the tablets to a powder even when consumption occurred within a few seconds.

### Example 8

In vitro dissolution studies to address the chewing issue.

This example demonstrates that controlled release matrix tablets containing carprofen release the drug at progressively faster rates when they are whole, halved, quartered, and crushed compared to whole in an in vitro dissolution test.

Controlled release matrix tablets containing carprofen were manufactured as follows: 67.3% lactose fast-flo, 15.0% Polyox Coagulant was blended in a Turbula blender for 20 min., then screened through a #40 mesh screen and blended for an additional 20 min.; 1.0% magnesium stearate was added and blended for an additional 3 min.; the result was compressed using a Manesty F press using 0.635×0.2175″ caplet shaped tooling at 600 mg target weight; 10 to 12 Kp hardness yielded tablets containing 100 mg of the active agent, carprofen.

TABLE 8
In vitro dissolution of whole, halved, quartered, and crushed
controlled release matrix tablets containing carprofen.
Values are percent (%) dissolved.

| | Whole (50 | Halved | Quartered | Crushed | Whole | Crushed |
|---|---|---|---|---|---|---|
| Lot 37255-41 | rpm) | (50 rpm) | (50 rpm) | (50 rpm) | (100 rpm) | (100 rpm) |
| 0.5 hr | 6 | 11 | 15 | 26 | 9 | 55 |
| 1 hr | 8 | 12 | 19 | 20 | 12 | 57 |
| 2 hr | 12 | 17 | 27 | 37 | 16 | 73 |
| 4 hr | 20 | 29 | 40 | 50 | 29 | 88 |
| 8 hr | 37 | 48 | 67 | 70 | 66 | 96 |
| 12 hr | 52 | 66 | 88 | 92 | 86 | 94 |
| 16 hr | 66 | 80 | 101 | 94 | 99 | 95 |
| 20 hr | 75 | 100 | 103 | 102 | 101 | 96 |
| 24 hr | 85 | 99 | 103 | 95 | 98 | 95 |

### Example 9

This example reports on in vivo studies with prototype controlled release matrix tablet formulations.

To access the potential loss of controlled release properties of matrix tablets, an in vivo experiment was conducted in dogs. Laboratory beagle dogs were dosed, in a cross over fashion, with the following formulations:

(A) flavored matrix tablets dosed in the “poke-down” or conventional fashion to ensure that they were not chewed.

(B) deliberately crushed matrix tablets dosed in capsules

(C) flavored controlled release matrix tablets offered to dogs as free choice

These studies and the pharmacokinetic analysis confirmed that the controlled release properties of matrix tablets were lost when the dogs were administered deliberately crushed tablets. Treatment C involved free choice acceptance of flavored matrix tablets by laboratory beagle dogs. Because of the low number of tablets consumed, it was not possible to obtain reliable pharmacokinetic date from Treatment C. Nonetheless, the deliberately crushed matrix tablets of Treatment B effectively simulated chewed tablets.

Based on the in vitro dissolution date of whole, halved, quartered, and crushed tablets and the observed chewing behavior of dogs consuming flavored tablets, it can be concluded that flavored controlled release matrix tablets, as configured according to the prior art for companion animals, will lead to a partial or full loss of the controlled release performance. This underscores the need for palatable controlled release formulations for companion animals that are resistant to chewing.

### Example 10

This example demonstrates the fabrication of pharmaceutically active agents in multiparticulate form in accordance with the invention, using wet granulation.

The following components were loaded in Lodige M20R high shear granulator (Lodige Process Technology, Inc., Marlton, N.J.) with 20L capacity using an impeller speed of 290 rpm: 30% carprofen, 4.93% pregelantinized starch, 60.14% lactose, and 4.93% sodium starch glycolate. The components were dry-mixed for 2 minutes followed by careful addition of an appropriate amount of deionized water to produce a wet granulation. The granulations were discharged and tray dried overnight at ˜50° C.

After overnight drying, a 20-140 mesh (106-850 μm) sieve cut was taken from the lot and used in fluid bed coating trials.

### Example 11

This example demonstrates the fabrication of pharmaceutically active agents in multiparticulate form in accordance with the invention by spray drying, and characterization of said multiparticulate.

The following formulations were spray dried using a Niro Portable Spray Drier (Niro, Inc. Columbia, Md.) equipped with a rotary atomizer. The feed rate ranged from 25 g/min to 100 g/min.

a) carprofen/acetone solution (20% solids)(Lot 34975-143)

b) carprofen/Eudragit/acetone solution (20% solids, 5:1 polymer:drug ratio) (Lot 34975-145-1 to -3)

c) carprofen/Eudragit/acetone solution (10% solids; 5:1 polymer:drug ratio) (Lot 35975-145-4 to -7)

The initial processing conditions for the spray drying runs were as given in Table 9:

| | | | | Inlet | |
|---|---|---|---|---|---|
| | | | | Chamber | Chamber |
| | | Inlet | Outlet | Gas | Pressure |
| | | Temp. | Temp. | Pressure | (mm |
| Solution | Lot No. | (° C.) | (° C.) | (mm water) | water) |
| Carprofen/ | 34975-143 | 130 | 75 | 45 | 20 |
| acetone | | | | | |
| (20% solids) | | | | | |
| Carprofen/ | 34975- | 110 | 65 | 44 | 10-15 |
| Eudragit/ | 145-1 to -3 | | | | |
| acetone | | | | | |
| (20% solids) | | | | | |
| Carprofen/ | 34975- | 110 | 65 | 44 | 10-15 |
| Eudragit/ | 145-4 to -7 | | | | |
| acetone | | | | | |
| (10% solids) | | | | | |

Physical characterization of the spray dried lots.

The particle size of the spray-dried particles was determined using a Malvern particle size analyzer (Malvern Instruments, Inc., Southborough, Mass.). The average particle size (denoted as mean) was in the range of about 20 μm to about 80 μm; the particular results were as follows:

| | |
|---|---|
| Carprofen alone | mean ˜ 18 μm |
| Carprofen/Eudragit (20% solids, 100 g/min feed) | mean ˜42 μm |
| Carprofen/Eudragit (20% solids, 50 g/min feed) | mean ˜69 μm |
| Carprofen/Eudragit (20% solids, 25 g/min feed) | mean ˜79 μm |
| Carprofen/Eudragit (10% solids, 25 g/min feed) | mean ˜22 μm |
| Carprofen/Eudragit (10% solids, 50 g/min feed) | mean ˜21 μm |
| Carprofen/Eudragit (10% solids, 25 g/min feed) | mean ˜28 μm |

The Differential Scanning Calorimetric (DSC) analysis of the spray dried particles showed no significant departures from the parent compound with the exception that the peak corresponding to the melting endotherm of carprofen was absent in mixtures with polymer present.

X-ray diffraction studies indicated that the drug was amorphous after spray-drying.

Scanning electron micrographs indicated that the particles were spherical in shape. Samples with polymers showed evidence of polymer “tails” and some hollow spheres were also noted. The particle sizes in the micrographs were consistent with the Malvern data.

### Example 12

This example shows the relationship between spray drier operating parameters and equipment configurations on resultant particle size (p.s.).

Several experiments were conducted to investigate the configuration of the spray dryer such as rotary atomizer versus two-fluid nozzle and two-point collection system. A two point collection system consists of a top collection port by means of a cyclone separator and a bottom collection system at the base of the spray-drying chamber by gravitation force acting on the particles. Also, several of the operating variables were studied such as nozzle pressure, feed rate, inlet temperature, and collection points.

A statistical analysis of the experimental results established that under certain conditions, it was feasible to manufacture particles having an average size of ˜100 μm in the spray drier. The larger particles (˜100 μm) are preferred over the smaller size particles (˜20 μm) because the larger particles are better accommodated for coating. As indicated in Table 10, which summarizes the experiments, the operating variables significantly affected the particle size, which ranged from about 15 μm to about 120 μm.

The experiments are summarized in Table 10.

TABLE 10
Spray Drier Optimization

| | | | | | | Part. | | | | | |
|---|---|---|---|---|---|---|---|---|---|---|---|
| | | | | | | size of | | | | | |
| | | | | | | max | Min. | | | Volume % | |
| | | | | | | vol. % | of | Max of | Range | of | |
| | | Feed | Inlet | Nozzle | Outlet | in main | main | main | of main | particles | Mean over |
| | Collection | Rate | Temp. | Press. | Temp. | peak | peak | peak | peak | in main | entire p.s. |
| Lot # | Point | (g/min) | (° C.) | (Bar) | (° C.) | (μm) | (μm) | (μm) | (μm) | peak | range (μm) |
| 34975- | Top | 200 | 170 | 2 | 58 | 13.04 | 0.75 | 68.18 | 67.43 | 87.56 | 17.5 |
| 163-1T | | | | | | | | | | | |
| 34975- | Bottom | 200 | 170 | 2 | 58 | 23.80 | 0.75 | 107.00 | 106.25 | 87.31 | 37.0 |
| 163-1B | | | | | | | | | | | |
| 34975- | Top | 50 | 115 | 1 | 65 | 11.22 | 0.75 | 79.24 | 78.49 | 87.38 | 15.0 |
| 163-2T | | | | | | | | | | | |
| 34975- | Bottom | 50 | 115 | 1 | 65 | 23.80 | 0.64 | 92.10 | 91.46 | 85.24 | 33.5 |
| 163-2B | | | | | | | | | | | |
| 34975- | Top | 300 | 170 | 1 | 66 | 15.10 | 0.75 | 79.24 | 78.49 | 85.65 | 40.1 |
| 163-3T | | | | | | | | | | | |
| 34975- | Bottom | 200 | 170 | 1 | 66 | 144.60 | 43.42 | 414.30 | 370.88 | 52.53 | 83.5 |
| 163-3B | | | | | | | | | | | |
| 35975- | Top | 50 | 115 | 0.5 | 74 | 13.04 | 0.75 | 58.66 | 57.91 | 88.29 | 21.0 |
| 163-4T | | | | | | | | | | | |
| 34975- | Bottom | 50 | 115 | 0.5 | 74 | 23.80 | 0.64 | 356.40 | 355.76 | 96.39 | 43.7 |
| 163-4B | | | | | | | | | | | |
| 34975- | Top | 200 | 170 | 0.5 | 66 | 17.62 | 0.75 | 92.10 | 91.35 | 89.44 | 25.6 |
| 163-5T | | | | | | | | | | | |
| 35975- | Bottom | 200 | 170 | 0.5 | 66 | 144.60 | 43.42 | 356.40 | 312.98 | 63.52 | 99.6 |
| 163-5B | | | | | | | | | | | |
| 34975- | Top | 50 | 115 | 2 | 68 | 11.22 | 0.75 | 68.18 | 67.43 | 77.09 | 31.8 |
| 163-6T | | | | | | | | | | | |
| 34975- | Bottom | 50 | 115 | 2 | 68 | 23.80 | 0.64 | 124.40 | 123.76 | 83.22 | 53.2 |
| 163-6B | | | | | | | | | | | |
| 34975- | Top | 100 | 150 | 0.3 | 83 | 17.62 | 0.75 | 68.18 | 67.43 | 91.94 | 19.4 |
| 163-7T | | | | | | | | | | | |
| 34975- | Bottom | 100 | 150 | 0.3 | 83 | 168.10 | 43.42 | 414.30 | 370.88 | 67.47 | 119.7 |
| 163-7B | | | | | | | | | | | |
| 34975- | Top | 200 | 180 | 0.3 | 83 | 20.47 | 0.75 | 79.24 | 78.49 | 90.53 | 21.9 |
| 163-8T | | | | | | | | | | | |
| 34975- | Bottom | 200 | 180 | 0.3 | 83 | 144.60 | 50.47 | 356.40 | 305.93 | 66.87 | 111.5 |
| 163-8B | | | | | | | | | | | |
| 34975- | Bottom | 200 | 170 | 0.4 | 71 | 23.80 | 0.64 | 92.10 | 91.46 | 75.07 | 56.0 |
| 163-9B | | | | | | | | | | | |

Note:
Feed rate, Inlet Temp and Nozzle Pressure were set during experiments
Main peak defined as peak with the region of “highest” volume % of particles
Volume % in main peak was defined as the addition of volume % over the range of the peak

### Example 13

This examples demonstrates an embodiment of a preparation of a pharmaceutically active agent in controlled release multiparticulate form as contemplated by the invention. In this example, the multiparticulate form was fabricated by wet granulation; coating was by fluidized bed.

The 20-140 mesh particles of Example 11 were coated in a Glatt GPCG-5 fluid bed coater (Glatt Air Techniques, Ramsey, N.J.). Two different coatings were applied.

A) Aquacoat coating (a 30% suspension of ethylcellulose polymer) at ˜27% w/w was applied to 1 kg of core granulation (the 20-140 mesh particles at Example 11) to obtain a sustained release coating.

B) Eudragit S100 coating at ˜15% w/w was applied (total coating weight of 23% by weight) to obtain a delayed release coating.

The fluid bed coating parameters used are given in Table 11.

TABLE 11
Coating parameters use din the fluid-bed coating runs

| Parameter | Aquacoat Coating | Eudragit S100 coating |
|---|---|---|
| Machine | Glatt GPCG-5 | Glass GPCG-5 |
| | with Wurster | with Wurster |
| | insert (Glatt | insert (Glass |
| | Air Techniques, | Air Techniques, |
| | Ramsey, New Jersey) | Ramsey, New Jersey |
| Partition insert | 20 mm | 10 mm |
| height | | |
| Air distribution | “B” plate with #80 | “A” plate with #80 |
| plate | twill screen | twill screen |
| Product | 40° +/− 5° C. | 30° C., range 28°-32° C. |
| temperature | | |
| Inlet air | 15% | 15% |
| flap setting | | |
| Inlet temperature | 50° C. or adjust | 40° C. or adjust |
| | to maintain | to maintain |
| | product temperature | product temperature |
| Spray rate | ˜30 g/min | ˜5-6 g/min |
| Nozzle | 1.2 mm | 0.8 mm |
| Pump | Gear pump | Gear pump |
| Atomization air | 1.3 Bar | 2 Bar |
| Air Volume | 12.5 M\hat{ }3/hr | 100 m\hat{ }3/hr |
| Exhaust Filter | 15 sec/3 sec | 15 sec/3 sec |
| Shake | | |
| Interval/Duration | | |
| Run Time | 15 min. | 158 min. |
| Coating Level | 27% w/w core | 23% w/w core |
| (assuming | | (15% Eudragit |
| 100% efficiency) | | polymer w/w core) |

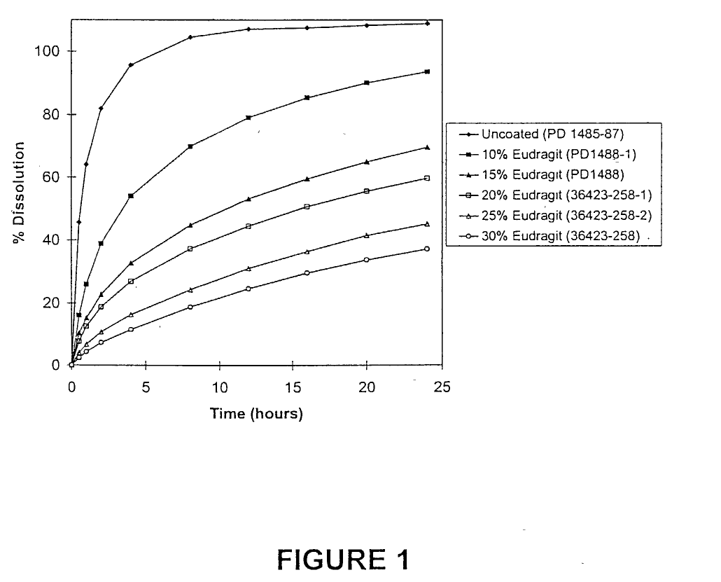

Although not required in the practice of the invention, some of the Eudragit-coated particles produced above were further coated with Eudragit S-100 using Glatt GPCG-1 fluid bed coater (Glatt Air Techniques, Ramsey, N.J.). Samples were taken at ˜20%, 25% and 30% w/w Eudragit S-100 solids. The dissolution of the Eudragit coated particles is given in FIG. 1.

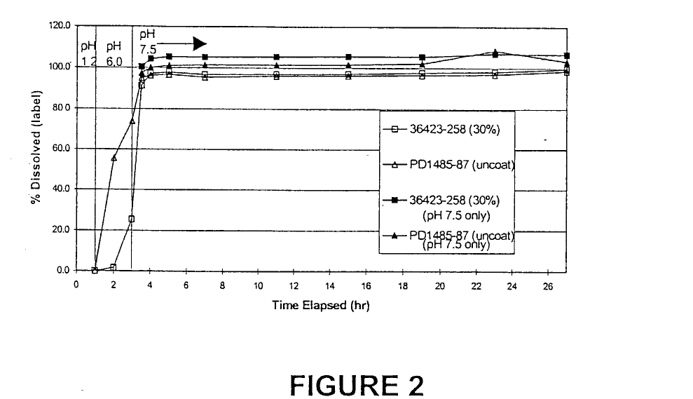

The 30% coated granulation was also tested at a pH-crossover dissolution test (FIG. 2) which was 1 hr in pH 1.2, followed by 2 hr in pH 6.0, and the remainder in pH 7.5. The data for pH 7.5 without exposure to the lower pHs is also shown. The results indicate no significant changes in the release profile between straight pH 7.5 and the pH-crossover dissolution.

### Example 14

Delayed Release Tablets.

To serve as a control, delayed release tablets containing 25 mg. carprofen (not in multiparticulate form) were manufactured using a powder blend that contained 12.5% carprofen, 43% microcrystalline cellulose, 43% dibasic calcium phosphate, a small amount of Yellow #10 Lake dye and 1% magnesium stearate. These tablet cores were coated in a side vented coating pan with Eudragit S100 to a 6% and 12% w/w polymer. The coating parameters are given in Table 12.

TABLE 12
Coating parameters for Eudragit S100 coating on tablets of Example 15.

| | |
|---|---|
| Machine | HCT-30 EP |
| Pump | Masterflex Peristaltic |
| Pan Speed | 20-25 rpm |
| Pan Load | 900 g (100 g active tablets) |
| Inlet Temperature | 47-50° C. (set at 50° C.) |
| Exhaust Temperature | 31-36° C. |
| Pump Speed | 5-7 rpm |
| Spray Rate | 4-7 g/min |
| Suspension applied | 972.1 g |
| Run Time | 156 min. |

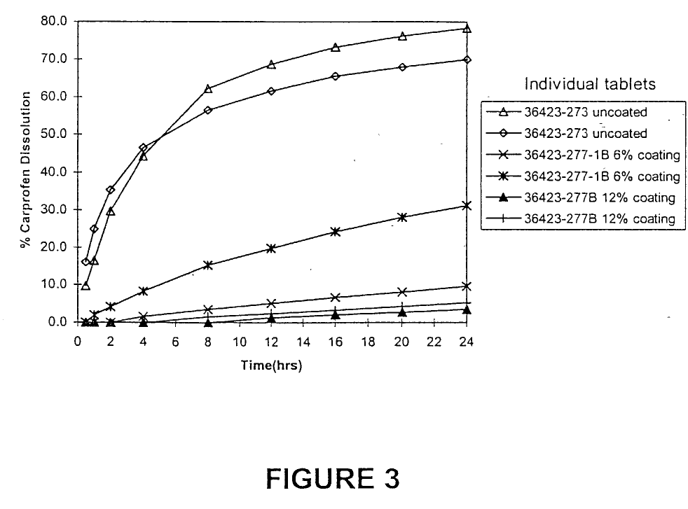

The dissolution results indicated that the 12% Eudragit S100 coating provided adequate enteric protection for the release of the drug. The dissolution results are given in FIG. 3.

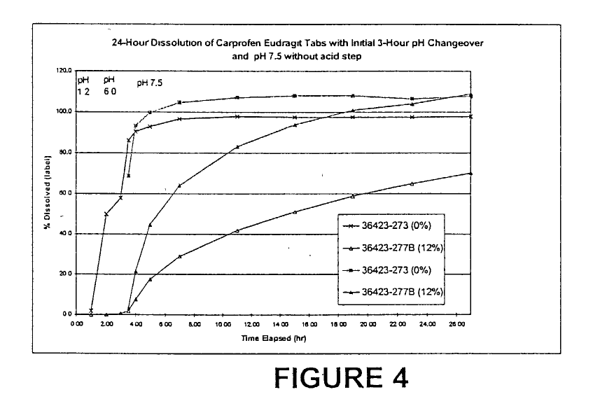

The 12% w/w Eudragit S100 coated tablets were also tested using the pH-changeover dissolution method and the results are shown in FIG. 4. The dissolution results showed that the coating provided adequate enteric protection.

### Example 15

A phase inversion process was used to manufacture carprofen-containing microcapsules. Carprofen and suitable solvents and polymer coatings were put through a microencapsulated process; filtered; dried in a tray drier (a fluid bed dryer can also be used); and sieved through a #20 mesh screen. The microcapsules were mixed with other inert ingredients and compressed to obtain 50 mg active in a 500 mg round tablet. The tablets were compressed with a Carver Press at ˜5 and 13 kp hardness.

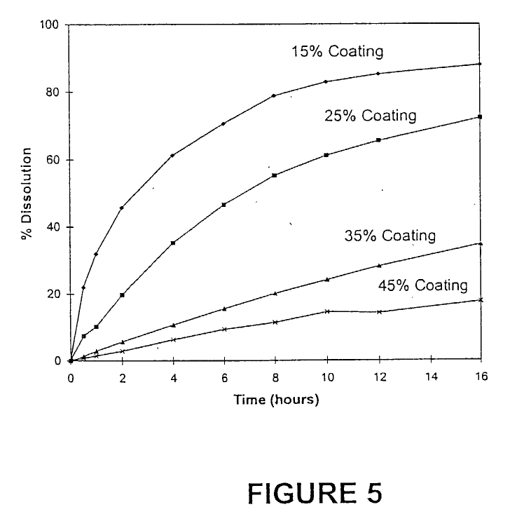

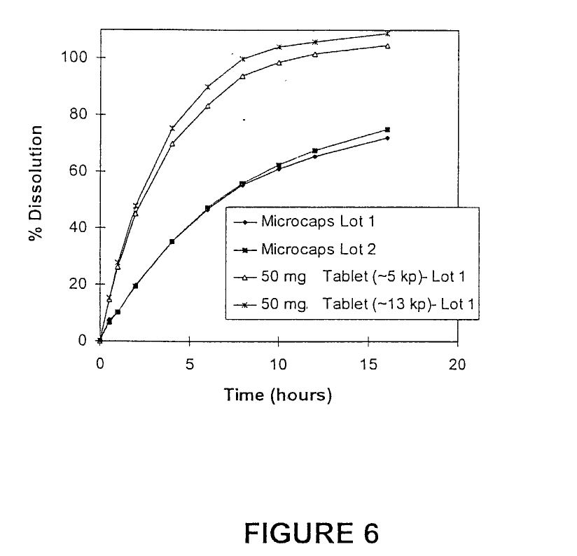

The dissolution results shown in FIG. 5 indicated that the coating was effective at slowing the carprofen release, and, as expected, release rate decreased with increasing coating level between 15% and 45% coating. Two lots of 25% coated microcapsules showed nearly identical release profiles (as shown in FIG. 6), indicating some degree of reproducibility of the coating process. There was a significant increase in release rates for all coating levels when tabletted, yet there was little difference in release profiles between “soft” (5 kp) and “hard” (13 kp) tablets. This indicates that the damage to the coating occurred during the initial compression of the blend.

### Example 16

Pharmaceutically active agents in multiparticulate form (core particle) of the invention by melt spray congealing.

Core particles are manufactured by a melt spray congealing (MSC) process as follows. The blend is prepared consisting of the pharmaceutically active agent, a natural or synthetic low melting (e.g., 50° to 80° C.) carrier (e.g., waxes such as carnauba wax, fatty acids such as stearic acid, mono-, di-, and tri-glycerides of fatty acids and their mixtures such as glyceryl monooleate, glyceryl momostearate, glyceryl paimitostearate, glyceryl behenate sold under the tradename Compritol® 888 ATO by Gattefosse S.A., France, paraffin, hydrogenated caster oil, lecithin, etc. and optionally (0% to about 15%) a surfactant (e.g. polyoxyethylene fatty acid esters, polysorbates, sorbitan esters, sorbitan fatty acid esters, or polyoxyethylene-polyoxyproplyene block copolymers sold under the tradenames of Lutrol® and Pluronic® or other amphiphilic waxy materials such as those sold under the tradename Gelucire® 44/14 or Gelucire® 50/13 by Gattefosse s.a., France. This blend is heated up to a suitable temperature in a melting tank or in an extruder. The hot mixture is atomized using a single-fluid or two-fluid spray nozzle or a centrifugal atomizer such as a rotating disk apparatus with a slotted wheel into a cooling chamber (e.g., a spray dryer). The cooler air in the chamber congeals the multiparticulates, which are sometimes referred to a microspheres.

### Example 17

Manufacture of Pharmaceutically Active Agents in Multiparticulate Form of the Invention (Core Particles) by Extrusion-spheronization

Core particles are manufactured by an extrusion-spheronization process as follows. A blend is prepared consisting of the drug (5% to 95% by weight in the dry mixture) and one or more binders and optionally a surfactant such as sodium laurly sulfate. The binders can be cellulose or natural gums, synthetic polymers, or microcrystalline cellulose. Microcrystalline cellulose (available in many different grades such as Avicel®, FMC Corporation grades PH101, PH102, RC-581, and CL-611), sodium carbosymethylcellulose, hydroxypropyl cellulose, hydroxypropylmethylcellulose, povidone, and pregelatinized starch. Water and water-alcohol mixture (called granulating liquid) is then added to the blend in a quantity sufficient to produce a wet mass of consistency that is suitable for the next step, which is extrusion. In the extruder (typically single crew or twin-screw extruder), the wet mass is forced through dies to form spaghetti-shaped cylinders. The cylinders or extrudate is then transformed into spherical or more-or-less spherical particles in a spheronizer. The spheronizer is essentially a bowl with a rapidly rotating bottom disc. The disc is machined to have crosshatched or radially-patterned grooves on its surface. In the final step, the core particles produced by extrusion-spheronization are dried in a conventional tray-dryer or a fluid-bed dryer.

### Example 18

Manufacture of Pharmaceutically Active Agents in Multiparticulate Form (Core Particles) of the Invention by Drug Layering.

Core particles are manufactured by a drug layering process as follows. Nonpareil seeds of a suitably small size are loaded into a fluid-bed unit or centrifugal granulator and a drug containing composition either in the solid form or as a suspension or solution is applied to the seeds. The drug containing composition contains a binder or alternatively, a binder solution is sprayed on the seeds while simultaneously applying the drug containing composition to essentially layer the drug onto the nonpareil seeds.

### Example 19

Coating of core particles to produce controlled release multiparticulate form of pharmaceutically active agent.

Core particles manufactured by any of the methods described in previous examples are coated to yield modified release of the active ingredient or drug. The coatings can be used to achieve delayed release (sometimes referred to as enteric coatings) or to achieve sustained release. Typically, the particles are coated in a fluid-bed coating unit fitted with a Wurster insert. The coating formulation can be either a suspension or a solution, using either aqueous or organic solvents or mixtures. The coating formulations typically contain the polymer, a plasticizer, and other formulation aids such as detackifiers, defoamers, surfactants, and the like. Polymers used to produce delayed release coatings are typically insoluble at low pH (range from 1 to about 5, typically found in the stomach) but are soluble at the higher pH (greater than 5.5, typically encountered in the small intestine). The polymers used for delayed release coatings include: cellulose acetate phthalate, hydroxypropylmethylcellulose phthalate, Eudragit L100-55, Eudragit S100, and mixtures of Eudragit L100/S100. Polymers used to produce sustained release coatings include: hydroxypropylmethylcellulose, ethylcellulose, Eudragit RL100, Eudragit RS100 and their mixtures, Eudragit S100, Eudragit NE30D, cellulose acetate, cellulose acetate butyrate, silicone, ethylcellulose dispersions sold under the tradenames of Aquacoat® FMC and Surrelease® Colorcon. Typical coating thicknesses for delayed release coatings are 20 to 30 um to yield the necessary mechanical stability and adequate dissolution performance. Typical coating thicknesses for sustained release coatings are in the range 5 to 50 um. In terms of weight (w/w core), the coatings can range from 5% to about 50 to 100% at the top end, and typically about 10 to 50%. Further information on polymeric coatings on multiparticulates are in the reference: Coating of multiparticulates using polymeric solutions, Formulation and Process Considerations, Klaus Lehmann, Rohm GmbH, Darmstadt, Germany in Multiparticulate oral drug delivery, edited by Isaac Ghebre-Sellassie, Marcel Dekker, Inc., 1994.

### Example 20

Manufacture of an Embodiment of the Palatable, Chewable, Controlled Release Composition of the Invention.

Multiparticulates as described in examples 17 through 19 and coated as described in Example 14, Example 16 or Example 20 and having the appropriate sustained or delayed release properties are blended with typical tablet excipients such as diluent, binders, lubricants, disintegrants, colors, and flavors. Typical diluents include: lactose, starch, mannitol, sorbitol, microcrystalline cellulose, dibasic calcium phosphate, sucrose calcium sulfate, calcium lactate, hydrolyzed starches, dextrose, amylose, etc. Typical binders are used in about 1% to about 20% by weight range and include, without limitation: acacia, cellulose derivatives, gelatin, glucose, polymethacrylates, povidone, sodium alginate, pregelantinized starch, etc. Typical disintegrants are used in the 1% to about 20% by weight range and include, without limitation: natural starch, sodium starch glycolate, pregelatinized starch, modified cornstarch, microcrystalline cellulose, alginates, gums, etc. Typical lubricants are used in quantities less than about 5% by weight and include, without limitation: magnesium, calcium, or sodium stearate, stearic acid, talc, polyethylene glycols, etc. Sometimes the tablet formulations also include antiadherents such as talc or cornstarch and glidants such as colloidal silicon dioxide. The tablets are made, e.g., by direct compression or by dry granulation (slugging, roller compaction, etc) or wet granulation. In addition to the abovementioned tablet ingredients, the flavored controlled release tablets also contain 1 to 30% of a palatability improving agent as described hereinbefore.

Reference for tablets: Pharmaceutical dosage forms: tablets, Volume 1, Edited by Herbert A. Lieberman, Leon Lachman, and Joseph B. Schwartz, Marcel Dekker, 1989, incorporated in its entirety herein by reference.

What is claimed is:

1. A palatable, chewable controlled release pharmaceutical composition for oral administration to a companion animal comprising:
a therapeutically effective amount of a pharmaceutically active agent in controlled release multiparticulate form; and
a palatability improving agent in an amount sufficient to make the pharmaceutical composition palatable to said companion animal.

2. The composition of claim 1 wherein said controlled release multiparticulate form has an average particle size of up to about 5000 μm.

3. The composition of claim 2 wherein said controlled release multiparticulate form has an average particle size of about 10 μm to about 5000 μm.

4. The composition of claim 3 wherein said controlled release microparticulate form has an average particle size of about 50 μm to about 2000 μm.

5. The composition of claim 4 wherein said controlled release microparticulate form has an average particle size of about 100 μm to about 1000 μm.

6. The composition of claim 1 wherein said controlled release microparticulate form is a sustained release microparticulate form, a delayed release microparticulate form or a pulsatile release microparticulate form.

7. The composition of claim 6 wherein said sustained release microparticulate form comprises particles coated with hydroxypropylmethyl cellulose, ethyl cellulose, Eudragit RL 100, Eudragit RS 100, mixtures of Eudragit RL 100/RS 100, Eudragit S100, Eudragit NE30D, cellulose acetate, cellulose acetate butyrate, silicone, ethylcellulose dispersions, or combinations thereof.

8. The composition of claim 7 wherein said particles are coated with ethyl cellulose.

9. The composition of claim 6 wherein said delayed release microparticulate form comprises particles coated with a pH sensitive material.

10. The composition of claim 9 wherein said pH sensitive material is cellulose acetate phthalate, hydroxypropylmethyl cellulose phthalate, Eudragit L100-55, Eudragit S100 and mixtures of Eudragit L100-55/S100, or combinations thereof.

11. The composition of claim 10 wherein said pH sensitive material is Eugragit S100.

12. The composition of claim 1 wherein said pharmaceutically active agent is selected from the group consisting of amebicides, trichomoacides, analgesics, anorexics, antiarthritics, anitbacterials, antibiotics, anticoagulants, antidepressants, anithistamines, antieoplastics, anti-Parkinsonism drugs, antipyretics, antispasmodics, antichoinergics, antiviral agents, cardiovascular drugs, contraceptives, diuretics, fertility agents hematinics, hormones, laxatives, parasympathetic agents, parasympathomometics, psyhostimulants, sedatives, sympathomimetics, anti-inflammatory agents, barbiturates, stimulants, tranquilizers and the like.

13. The composition of claim 1 wherein said palatability improving agent is meat-derived, non-meat derived, fish-derived, non-fish derived, yeast, yeast hydrozalate or combinations thereof.

14. The composition of claim 13 wherein said palatability improving agent is selected from the group consisting of artificial egg, artificial beef, artificial poultry, artificial fish, dairy-based palatability improving agents, natural herbs and spices and combinations thereof.

15. The composition of claim 13 wherein said palatability improving agent is present in an amount of about 0.025% to about 99% by weight of said pharmaceutical composition.

16. The composition of claim 15 wherein said palatability improving agent is present in an amount of about 0.75% to about 50% by weight of said pharmaceutical composition.

17. The composition of claim 16 wherein said palatability improving agent is present in an amount of about 1% to about 25% by weight of said pharmaceutical composition.

18. A palatable, chewable controlled release pharmaceutical composition for oral administration to a companion animal comprising:
a therapeutically effective amount of a pharmaceutically active agent in multiparticulate form comprising particles of said pharmaceutically active agent having an average particle size of up to about 5000 μm, said particles being coated with hydroxypropylmethyl cellulose, ethyl cellulose, Eudragit RL100, Eudragit RS100, mixtures of Eudragit RL100/RS 100, Eudragit NE30D, cellulose acetate butyrate, silicone, ethylcellulose dispersions, or combinations thereof, cellulose acetate phthalate, hydroxypropylmethyl cellulose phthalate, Eudragit L100-55, Eudragit S100 and mixtures of Eudragit L100-55/S100, or combinations thereof, said coating being present in an amount of about 5% to about 100% by weight of said pharmaceutical composition; and
a palatability improving agent that is meat-derived, non-meat derived, fish-derived, non-fish derived, yeast or yeast hydrozalate, said palatability improving agent present in an amount of about 0.025% to about 99% by weight of said pharmaceutical composition.

19. The composition of claim 18 wherein said pharmaceutically active agent comprises particles of an anti-inflammatory agent having an average particle size of about 100 μm to about 1000 μm, said coating present in an amount of about 10% to about 50% by weight of said pharmaceutical composition; and
said palatability improving agent is present in an amount of about 1% to about 5% by weight of said pharmaceutical composition.

20. The composition of claim 19 wherein said anti-inflammatory agent is an NSAID.

21. The composition of claim 20 wherein said NSAID is carprofen, and said coating is polymeric ethylcellulose or an acrylic polymer that is an anionic copolymer made from methacrylic acid and methacrylate.

22. The composition of claim 1 wherein said pharmaceutical composition for oral administration is in a dosage form whose size and shape are suitable for poke down administration to a dog or cat.

23. The composition of claim 1 wherein said pharmaceutical composition for oral administration is in a dosage form that has means for enabling division of said dosage form into smaller sizes.

24 A process for preparing a palatable, chewable controlled release pharmaceutical composition for oral administration to a companion animal comprising:
preparing a therapeutically effective amount of a pharmaceutically active agent in the form of particles having an average particle size of up to about 5000 μm;
coating said particles with a delayed release, sustained release or pulsatile release material in an amount of about 5% to about 100% by weight of the pharmaceutical composition;
admixing a palatability improving agent to said coated particles in an amount of about 0.025% to about 99% by weight of said pharmaceutical composition; and
forming said admixture into a shape suitable for oral administration to a companion animal.

25. The process of claim 22 wherein the preparing of said particles is by balling, spray congealing, cyropelletization, melt-spray congealing, spray-drying, coacervation, interfacial polymerization, phase separation, dry granulation, wet granulation, extrusion-spheronization, drug-layering or combinations thereof; and said coating is by fluidized bed, pan coating or combinations thereof.
